(19) United States
(12) Patent Application Publication
(10) Pub. No.: US 2012/0035268 A1
(43) Pub. Date: Feb. 9, 2012

(54) SPHINGO-GUANIDINES AND THEIR USE AS INHIBITORS OF SPHINGOSINE KINASE

(76) Inventors: Zdzislaw M. Szulc, Charleston, SC (US); Alicja Bielawska, Charleston, SC (US); Lina M. Obeld, Sullivan's Island, SC (US); Yusuf A. Hannun, Sullivan's Island, SC (US); James Norris, Mount Pleasant, SC (US); Liu Xiang, Mount Pleasant, SC (US)

(21) Appl. No.: 13/142,421

(22) PCT Filed: Dec. 28, 2009
(86) PCT No.: PCT/US09/69583

§ 371 Date: Sep. 29, 2011

Related U.S. Application Data
(60) Provisional application No. 61/141,464, filed on Dec. 30, 2008.

(51) Int. Cl. A61K 31/155; C12N 9/99; A61P 35/00; A61P 35/04; A61P 17/02; A61P 9/00; A61P 29/00; A61P 37/00; A61P 11/06; A61P 17/00; C07C 279/08; A61P 9/10

(52) U.S. Cl. 514/634; 564/240; 435/184

(57) ABSTRACT

The presently disclosed subject matter provides compounds of the formula: (1) and pharmaceutically acceptable salts thereof, wherein R₁, R₂, R₃, R₄, R₅, R₆, R₇, and R₈ are as defined herein. Also disclosed are methods for making the compounds of the formula as set forth hereinabove, their use in inhibiting sphingosine kinase, and their use in the treatment and/or prevention of diseases and/or conditions associated with undesirable ceramidase or sphingosine kinase activity, including, but not limit cancer, cancer metastasis, atherosclerosis, stenosis, inflammation, immunological disorders, asthma, atopic dermatitis, wound healing, and other proliferative diseases.

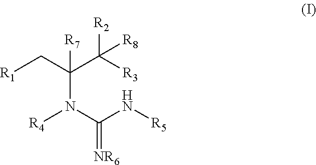

### CROSS REFERENCE TO RELATED APPLICATION

This application claims the benefit of U.S. Provisional Patent Application Ser. No. 61/141,464, filed Dec. 30, 2008, the disclosure of which is incorporated herein by reference in its entirety.

### GOVERNMENT INTEREST

This presently disclosed subject matter was made with U.S. Government support under Grant Nos. R01 GM062887 and P01 CA097132 awarded by the National Institute of General Medical Science and the National Cancer Institute of the National Institutes of Health. Thus, the U.S. Government has certain rights in the presently disclosed subject matter.

### TECHNICAL FIELD

The presently disclosed subject matter relates to sphingo-guanidines and their use as inhibitors of sphingosine kinase. The compounds can also be used to treat or prevent diseases related to undesirable sphingosine kinase activity, including cancer and other proliferative diseases.

### BACKGROUND

Sphingolipids act as signaling molecules in the regulation of fundamental cellular responses, such as cell death, differentiation and proliferation. See Pettus et al., (2002) Biochim. Biophys. Acta., 1585, 114; Maceyka et al., (2002) Biochim. Biophys. Acta., 1585, 193; Taha et al., (2006) Biochim. Biophys. Acta., 1758, 2027; Ogretmen and Hannun, (2004) Nat. Rev. Cancer, 4, 604; and Huwiler and Zangemeister-Wittke, (2007) Oncology Hematol., 63, 150-159. In particular, the cellular balance between ceramide (Cer) and sphingosine 1-phosphate (S1P) appears to affect a cell's decision to either undergo apoptosis or to proliferate, two events which are implicated in tumor developments and growth. Whereas Cer possesses pro-apoptotic capacity in many cell types, S1P acts as a counter player able to induce cell proliferation and protect cells from undergoing apoptosis.

More particularly, S1P can act as a potent mitogen and natural ligand to extracellular EDG receptors, which control calcium mobilization, regulation of growth, cytoskeletal organization, differentiation, migration and angiogenesis. See Lee et al., (1998) Science, 279, 1552-1555; Maceyka et al., (2002) Biochim. Biophys. Acta., 1585, 193-201; Hla, (2003) Pharmacol. Res., 47, 401-407; Watterson et al., (2003) Prog. Lipid Res., 42, 344-357; Novgorodov et al. (2007) FASEB J., 21, 1503-1514; and Pettus et al., (2003) FASEB J., 17, 1411-1421. Further, S1P is abundantly present in blood platelets and appears to act in blood vessel formation. See Yatomi et al., (1995) Blood, 86, 193-202; English et al., (2000) FASEB J., 14, 2255-2265; and Takeya et al., (2003) Blood, 102, 1693-1700. S1P has been implicated in the pathophysiology of immunulogical and inflammatory disorders, wound healing, atherosclerosis and cancer. See Pettus et al., (2003) FASEB J., 17, 1411-1421; Johnson et al., (2003) J. Biol. Chem., 278, 34541-34547; French et al., (2003) Cancer Res., 63, 5962-5969; and Kim et al., (2006) Bioorg. Med. Chem., 13, 3475.

In the cell, ceramidase (CDase) produces sphingosine (Sph) and fatty acids from Cer. The Sph can then be phosphorylated by sphingosine kinase (SK) to form S1P or be used as a substrate for ceramide synthases to resynthesize new Cers. See Bielawska et al., (2008) Bioorg, Med. Chem., 16, 2008. Two isoforms of SK have been reported: sphingosine kinase 1 (SK1) and sphingosine kinase 2 (SK2). See Maceyka et al., (2002) Biochim. Biophys. Acta., 1585, 193; and Taha et al., (2006) Biochim. Biophys. Acta., 1758, 2027. Although both produce S1P, it appears that SK1 and SK2 can serve opposite cellular functions. SK1 appears to mainly promote proliferation and migration, whereas SK2 favors a proapoptotic response. Tipping the cellular balance in favor of Cer production, for example, by inhibiting CDase or certain SK activities has potential to support its pro-apoptotic action and represents a promising approach to effective cancer therapy. See Ogretmen and Hannun, (2004) Nat. Rev. Cancer, 4, 604; and Huwiler and Zangemeister-Wittke, (2007) Oncology Hematol., 63, 150-159.

Accordingly, there appears to be an ongoing need for compounds that can effectively inhibit SK, particularly those that can inhibit SK1 selectively in varied tumor models and at low concentrations. There also remains a need for further water soluble SK inhibitors, classes of SK inhibitors that include compounds with varied levels of hydrophobicity, and/or fully water soluble SK inhibitors.

### SUMMARY

The presently disclosed subject matter provides, in some embodiments, a compound of Formula (I):

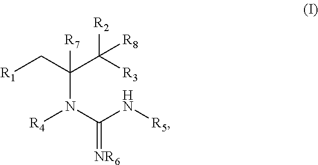

wherein:

R₁ is selected from the group comprising H, alkyl, OH, (═O), (═N—OH), and SH;

R₂ is selected from the group comprising H, alkyl, OH, SH, (═O), and (═N—OH);

R₃ is selected from the group comprising H, alkyl, substituted alkyl, unsaturated alkyl, aryl, substituted aryl, aralkyl, OH, alkoxyl, aryloxyl, aralkoxyl, amino, aminoalkyl, an aminoacid moiety, and a peptidyl moiety;

R₄ and R₅ are independently selected from the group comprising H, C(═NH)—NH₂, acyl, alkyl, substituted alkyl, aralkyl, aryl, and substituted aryl;

R₆ is selected from the group comprising H, CN, alkyl, substituted alkyl, aryl, substituted aryl, aralkyl, and acyl;

R₇ is selected from the group comprising H, alkyl, substituted alkyl, aralkyl, aryl, and substituted aryl; and

R₈ is present or absent, and when present is selected from the group comprising H, alkyl, substituted alkyl, aralkyl, aryl, and substituted aryl;

or a pharmaceutically acceptable salt thereof.

In some embodiments, R₁ is selected from OH and H. In some embodiments, R₂ is selected from OH, (═O), and H. In some embodiments, R₁ and R₂ are each OH.

In some embodiments, R₃ is selected from H, C₁₅ fully saturated alkyl, C₁₅ alkenyl, C₁₅ alkynyl, aralkyl, and phenyl. In some embodiments, R₄ is H. In some embodiments, R₅ is H.

In some embodiments, the compound comprises a pharmaceutically acceptable salt and has a structure of Formula (Ia):

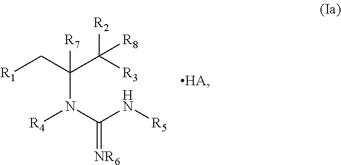

wherein:

R₁ is selected from the group comprising H, alkyl, OH, (═O), (═N—OH), and SH;

R₂ is selected from the group comprising H, alkyl, OH, SH, (═O), and (═N—OH);

R₃ is selected from the group comprising H, alkyl, substituted alkyl, unsaturated alkyl, aryl, substituted aryl, aralkyl, OH, alkoxyl, aryloxyl, aralkoxyl, amino, aminoalkyl, an aminoacid moiety, and a peptidyl moiety;

R₄ and R₅ are independently selected from the group comprising H, C(═NH)—NH₂, acyl, alkyl, substituted alkyl, aralkyl, aryl and substituted aryl;

R₆ is selected from the group comprising H, CN, alkyl, substituted alkyl, aryl, substituted aryl, aralkyl, and acyl;

R₇ is selected from the group comprising H, alkyl, substituted alkyl, aralkyl, aryl, and substituted aryl;

R₈ is present or absent, and when present is selected from the group comprising H, alkyl, substituted alkyl, aralkyl, aryl, and substituted aryl; and

A is selected from the group comprising bromide, chloride, sulfate, acetate, dichloroacetate, benzoate, and tartrate.

In some embodiments, the compound is a compound of Formula (Ia) wherein A is chloride.

In some embodiments, the compound is selected from the group comprising D-erythro-2-guanidino-sphingosine hydrochloride and L-erythro-2-guanidino-sphinogosine hydrochloride.

In some embodiments, the presently disclosed subject matter provides a method of inhibiting sphingosine kinase, the method comprising contacting a sample comprising sphingosine kinase with an effective amount of a compound of Formula (I). In some embodiments, the compound selectively inhibits sphingosine kinase 1 (SK1). In some embodiments, the compound is essentially completely water soluble. In some embodiments, the sample is an in vitro sample. In some embodiments, the compound inhibits sphingosine kinase with a 50% inhibitory concentration (IC₅₀) of less that about 1.0 μM.

In some embodiments, the presently disclosed subject matter provides a method of treating or preventing a disease or disorder associated with undesirable ceramidase, ceramidase-related, or sphingosine kinase activity in a subject, the method comprising administering to the subject an effective amount of a compound of Formula (I). In some embodiments, the disease or disorder is selected from the group comprising cancer, cancer metastasis, atherosclerosis, stenosis, inflammation, an immunological disorder, asthma, atopic dermatitis, wound healing, and other proliferative disorders. In some embodiments, the subject is a mammalian subject. In some embodiments, the compound of Formula (I) is administered to the subject in a pharmaceutical formulation comprising the compound of Formula (I) and a pharmaceutically acceptable carrier. In some embodiments, the compound of Formula (I) is essentially completely water soluble.

In some embodiments, the presently disclosed subject matter provides a method of treating cancer, the method comprising administering to a subject in need of treatment thereof an effective amount of a compound of Formula (I). In some embodiments, the cancer is selected from the group consisting of breast cancer, prostate cancer, and non small cell lung cancer. In some embodiments, treating cancer comprises preventing the metastasis of a cancer.

It is an object of the presently disclosed subject matter to provide compounds for inhibiting sphingosine kinase and for use in treating or preventing diseases and disorders related to undesirable ceramidase, ceramidase-related, or sphingosine kinase activity, including cancer and other proliferative diseases.

An object of the presently disclosed subject matter having been stated hereinabove, and which is achieved in whole or in part by the presently disclosed subject matter, other objects will become evident as the description proceeds when taken in connection with the accompanying drawings as best described hereinbelow.

### BRIEF DESCRIPTION OF THE DRAWINGS

FIG. 1 is a graph comparing the in vitro inhibition of purified sphingosine kinase 1 (SK1) by 0-50 μM D-erythro-2-N-(1′-carboxamidino)-sphingosine hydrochloride (LCL146, darkly shaded diamonds), L-erythro-2-N-(1′-carboxamidino)-sphingosine hydrochloride (LCL351, lightly shaded squares) or D-erythro-N,N-dimethyl-sphingosine (N,N-DMS, light colored triangles). Inhibition of SK1 is measured as a percentage of control (uninhibited SK1).

FIG. 2A is a graph showing the inhibitory effects of 24 hour pretreatment with 0-25 μM D-erythro-2-N-(1′-carboxamidino)-sphingosine hydrochloride (LCL146, darkly shaded diamonds) or L-erythro-2-N-(1′-carboxamidino)-sphingosine hydrochloride (LCL351, lightly shaded squares) on sphingosine kinase 1 (SK1) activity in human lung cancer cells (H650 cells).

FIG. 2B is a graph showing the time dependence of the inhibitory effects of 12.5 μM D-erythro-2-N-(1′-carboxamidino)-sphingosine hydrochloride (LCL146, darkly shaded diamonds) or L-erythro-2-N-(1′-carboxamidino)-sphingosine hydrochloride (LCL351, lightly shaded squares) on sphingosine kinase 1 (SK1) activity in human lung cancer cells (H650 cells).

FIG. 3A is a graph showing the inhibitory effects of 24 hour pretreatment with 0-25 μM D-erythro-2-N-(1′-carboxamidino)-sphingosine hydrochloride (LCL146, darkly shaded diamonds) or L-erythro-2-N-(1′-carboxamidino)-sphingosine hydrochloride (LCL351, lightly shaded squares) on sphingosine kinase 1 (SK1) activity in human lung cancer cells (H2405 cells).

FIG. 3B is a graph showing the time dependence of the inhibitory effects of 12.5 μM D-erythro-2-N-(1′-carboxamidino)-sphingosine hydrochloride (LCL146, darkly shaded diamonds) and L-erythro-2-N-(1′-carboxamidino)-sphingosine hydrochloride (LCL351, shaded squares) on sphingosine kinase 1 (SK1) activity in human lung cancer cells (H2405 cells).

FIG. 4A is a bar graph showing the inhibitory effects of 24 pretreatment with D-erythro-2-N-(1′-carboxamidino)-sphingosine hydrochloride (LCL146, middle bar, 10 μM) or L-erythro-2-N-(1′-carboxamidino)-sphingosine hydrochloride (LCL351, right-hand bar, 10 μM) on sphingosine 1-phosphate (S1P) formation from L-erythro-sphingosine (L-e-Sph) in human breast cancer (MCF7) cells. S1P formation in non-pretreated cells (to which only L-e-Sph had been added) is shown in the bar on the left-hand side.

FIG. 4B is a bar graph showing the inhibitory effects of D-erythro-2-N-(1′-carboxamidino)-sphingosine hydrochloride (LCL146, darkly shaded bars, 10 μM) or L-erythro-2-N-(1′-carboxamidino)-sphingosine hydrochloride (LCL351, unshaded bars, 10 μM) on sphingosine 1-phosphate (S1P) formation from L-erythro-sphingosine (L-e-Sph) in human breast cancer (MCF7) cells when the LCL146 or LCL351 is added at the same time as the L-e-Sph. Data is shown for 2 hour, 10 hour, and 24 hours of simultaneous treatment with the inhibitor and the L-e-Sph. For comparison, S1P formation in cells to which only L-e-Sph had been added is shown in the bars with the lighter level of shading.

FIG. 5A is a graph showing the inhibitory effects of 24 hour pretreatment with 0-34 μM D-erythro-2-N-(1′-carboxamidino)-sphingosine hydrochloride (LCL146, darkly shaded diamonds) or L-erythro-2-N-(1′-carboxamidino)-sphingosine hydrochloride (LCL351, lightly shaded squares) on cell growth of human breast cancer (MCF7) cells after 48 hours. Cell growth is reported as a percentage of the growth observed in control cells, which had not been exposed to inhibitors.

FIG. 5B is a bar graph showing that guanidino-sphingosine compounds enhance the inhibitory effects of D-erythro-sphingosine (D-e-Sph) and L-erythro-sphingosine (L-e-Sph) on human breast cancer (MCF7) cells at 10 hours (lightly shaded bars) and 24 hours (darkly shaded bars). The results are provided as a percentage of cell growth observed in control cells that were not treated with guanidino-sphingosine or sphingosine compounds. Data is given for cells treated with D-erythro-2-N-(1′-carboxamidino)-sphingosine hydrochloride (LCL146, 10 μM) or L-erythro-2-N-(1′-carboxamidino)-sphingosine hydrochloride (LCL351, 10 μM) alone and for cells treated with D-e-Sph or L-e-Sph alone, as well as for cells treated with combinations of D-e-Sph and LCL146 or LCL351 and combinations of L-e-Sph and LCL146 or LCL351, as indicated at the bottom of the graph.

FIG. 6A is a graph showing the effects of 0-25 μM D-erythro-2-N-(1′-carboxamidino)-sphingosine hydrochloride (LCL146, darkly shaded diamonds) or L-erythro-2-N-(1′-carboxamidino)-sphingosine hydrochloride (LCL351, lightly shaded squares) on cell viability of human non small cell lung cancer (H650) cells after 48 hours.

FIG. 6B is a graph showing the effects of 0-25 μM D-erythro-2-N-(1′-carboxamidino)-sphingosine hydrochloride (LCL146, darkly shaded diamonds) or L-erythro-2-N-(1′-carboxamidino)-sphingosine hydrochloride (LCL351, lightly shaded squares) on cell viability of human non small cell lung cancer (H2405) cells after 48 hours.

FIG. 7 is a graph of showing the concentration dependent elevation of endogenous C₁₆-ceramide caused by 0-25 μM D-erythro-2-N-(1′-carboxamidino)-sphingosine hydrochloride (LCL146, darkly shaded diamonds) or L-erythro-2-N-(1′-carboxamidino)-sphingosine hydrochloride (LCL351, lightly shaded squares) in human breast cancer (MCF7) cells.

FIG. 8 is a bar graph showing the effects of 5 μM L-erythro-2-N-(1′-carboxamidino)-sphingosine hydrochloride (LCL351) on cell migration in human prostate (DU145) cells that overexpress acid ceramidase. NT represents non-treated cells.

### DETAILED DESCRIPTION

The presently disclosed subject matter will now be described more fully hereinafter with reference to the accompanying Examples, in which representative embodiments are shown. The presently disclosed subject matter can, however, be embodied in different forms and should not be construed as limited to the embodiments set forth herein. Rather, these embodiments are provided so that this disclosure will be thorough and complete, and will fully convey the scope of the embodiments to those skilled in the art.

All references cited herein, including all patents, patent applications, database entries, and journal articles, are incorporated herein by reference in their entireties and for all purposes to the same extent as if each individual patent, patent application, database entry, or journal article was specifically and individually indicated to be incorporated by reference in its entirety for all purposes.

### I. Definitions

All technical and scientific terms used herein, unless otherwise defined below, are intended to have the same meaning as commonly understood by one of ordinary skill in the art. References to techniques employed herein are intended to refer to the techniques as commonly understood in the art, including variations on those techniques or substitutions of equivalent techniques that would be apparent to one of skill in the art. While the following terms are believed to be well understood by one of ordinary skill in the art, the following definitions are set forth to facilitate explanation of the presently disclosed subject matter.

Throughout the specification and claims, a given chemical formula or name shall encompass all optical and stereoisomers, as well as racemic mixtures where such isomers and mixtures exist, unless otherwise specified.

Following long-standing patent law convention, the terms “a”, “an”, and “the” refer to “one or more” when used in this application, including the claims. Thus, the phrase “a compound” refers to one or more compounds.

The term “comprising”, which is synonymous with “including,” “containing,” or “characterized by” is inclusive or open-ended and does not exclude additional, unrecited elements or method steps. “Comprising” is a term of art used in claim language which means that the named elements are essential, but other elements can be added and still form a construct within the scope of the claim.

As used herein, the phrase “consisting of” excludes any element, step, or ingredient not specified in the claim. When the phrase “consists of” appears in a clause of the body of a claim, rather than immediately following the preamble, it limits only the element set forth in that clause; other elements are not excluded from the claim as a whole.

As used herein, the phrase “consisting essentially of” limits the scope of a claim to the specified materials or steps, plus those that do not materially affect the basic and novel characteristic(s) of the claimed subject matter.

With respect to the terms “comprising”, “consisting of”, and “consisting essentially of”, where one of these three terms is used herein, the presently disclosed and claimed subject matter can include the use of either of the other two terms. The term “about”, as used herein when referring to a measurable value such as an amount of weight, time, dose, etc., is meant to encompass variations of in some embodiments ±20% or ±10%, in some embodiments ±5%, in some embodiments ±1%, and in some embodiments ±0.1% from the specified amount, as such variations are appropriate to perform the disclosed methods.

As used herein the term “alkyl” refers to C₁₋₂₀ inclusive, linear (i.e., “straight-chain”), branched, or cyclic, saturated or at least partially and in some cases fully unsaturated (i.e., alkenyl and alkynyl) hydrocarbon chains, including for example, methyl, ethyl, propyl, isopropyl, butyl, isobutyl, tert-butyl, pentyl, hexyl, octyl, ethenyl, propenyl, butenyl, pentenyl, hexenyl, octenyl, butadienyl, propynyl, butynyl, pentynyl, hexynyl, heptynyl, and alkenyl groups. “Branched” refers to an alkyl group in which a lower alkyl group, such as methyl, ethyl or propyl, is attached to a linear alkyl chain. “Lower alkyl” refers to an alkyl group having 1 to about 8 carbon atoms (i.e., a C₁₋₈ alkyl), e.g., 1, 2, 3, 4, 5, 6, 7, or 8 carbon atoms. “Higher alkyl” refers to an alkyl group having about 10 to about 20 carbon atoms, e.g., 10, 11, 12, 13, 14, 15, 16, 17, 18, 19, or 20 carbon atoms. In certain embodiments, “alkyl” refers, in particular, to C₁₋₈ straight-chain alkyls. In other embodiments, “alkyl” refers, in particular, to C₁₋₈ branched-chain alkyls. In some embodiments, “alkyl” refers to a C₁₅ saturated alkyl or a C₁₅ alkenyl or alkynyl group.

Alkyl groups can optionally be substituted (a “substituted alkyl”) with one or more alkyl group substituents, which can be the same or different. The term “alkyl group substituent” includes but is not limited to alkyl, substituted alkyl, halo, arylamino, acyl, hydroxyl, aryloxyl, alkoxyl, alkylthio, arylthio, aralkyloxyl, aralkylthio, carboxyl, alkoxycarbonyl, oxo, and cycloalkyl. There can be optionally inserted along the alkyl chain one or more oxygen, sulfur or substituted or unsubstituted nitrogen atoms, wherein the nitrogen substituent is hydrogen, lower alkyl (also referred to herein as “alkylaminoalkyl”), or aryl.

Thus, as used herein, the term “substituted alkyl” includes alkyl groups, as defined herein, in which one or more atoms or functional groups of the alkyl group are replaced with another atom or functional group, including for example, alkyl, substituted alkyl, halogen, aryl, substituted aryl, alkoxyl, hydroxyl, nitro, amino, alkylamino, dialkylamino, sulfate, and mercapto.

As used herein “alkenyl” refers to an alkyl group as defined hereinabove that contains at least one carbon-carbon double bond (e.g., —CH═CH—). In some embodiments, the alkenyl group is a vinyl alkyl group and has the structure —CH═CHR, wherein R is alkyl.

“Alkynyl” as used herein refers to an alkyl group as defined hereinabove, that contains at least one carbon-carbon triple bond. In some embodiments, the alkynyl group is an acetylene alkyl group and has the structure —C≡CR, wherein R is alkyl.

The term “aryl” is used herein to refer to an aromatic substituent that can be a single aromatic ring, or multiple aromatic rings that are fused together, linked covalently, or linked to a common group, such as, but not limited to, a methylene or ethylene moiety. The common linking group also can be a carbonyl, as in benzophenone, or oxygen, as in diphenylether, or nitrogen, as in diphenylamine. The term “aryl” specifically encompasses heterocyclic aromatic compounds. The aromatic ring(s) can comprise phenyl, naphthyl, biphenyl, diphenylether, diphenylamine and benzophenone, among others. In particular embodiments, the term “aryl” means a cyclic aromatic comprising about 5 to about 10 carbon atoms, e.g., 5, 6, 7, 8, 9, or 10 carbon atoms, and including 5- and 6-membered hydrocarbon and heterocyclic aromatic rings.

The aryl group can be optionally substituted (a “substituted aryl”) with one or more aryl group substituents, which can be the same or different, wherein “aryl group substituent” includes alkyl, substituted alkyl, aryl, substituted aryl, aralkyl, hydroxyl, alkoxyl, aryloxyl, aralkyloxyl, carboxyl, acyl, halo, nitro, alkoxycarbonyl, aryloxycarbonyl, aralkoxycarbonyl, acyloxyl, acylamino, aroylamino, carbamoyl, alkylcarbamoyl, dialkylcarbamoyl, arylthio, alkylthio, alkylene, and —NR′R″, wherein R′ and R″ can each be independently hydrogen, alkyl, substituted alkyl, aryl, substituted aryl, and aralkyl.

Thus, as used herein, the term “substituted aryl” includes aryl groups, as defined herein, in which one or more atoms or functional groups of the aryl group are replaced with another atom or functional group, including for example, alkyl, substituted alkyl, halogen, aryl, substituted aryl, alkoxyl, hydroxyl, nitro, amino, alkylamino, dialkylamino, sulfate, and mercapto.

Specific examples of aryl groups include, but are not limited to, cyclopentadienyl, phenyl, furan, thiophene, pyrrole, pyran, pyridine, imidazole, benzimidazole, isothiazole, isoxazole, pyrazole, pyrazine, triazine, pyrimidine, quinoline, isoquinoline, indole, carbazole, and the like.

“Aralkyl” refers to a group including a combination of alkyl and aryl moieties. Thus, the aralkyl group can be an aryl-alkyl-, alkyl-aryl-, alkyl-aryl-alkyl-, or aryl-alkyl-aryl- group wherein aryl and alkyl are as previously described, and can include substituted aryl and substituted alkyl. Exemplary aralkyl groups include benzyl, phenylethyl, and naphthylmethyl.

The terms “alkoxy” or “alkoxyl” refer to the group —O-alkyl, wherein alkyl is as defined hereinabove. Exemplary alkoxy groups include methoxy, ethoxy, propoxy, isopropoxy, butoxy, tert-butoxy, and the like.

The terms “aryloxy” and “aryloxyl” refer to the —O-aryl group, wherein aryl is as defined hereinabove. An exemplary aryloxyl group is phenoxy.

The terms “aralkoxy” and aralkoxyl” refer to the —O-aralkyl group, wherein aralkyl is as defined hereinabove. An exemplary aralkoxy group is benzyloxy.

As used herein, the term “acyl” refers to an organic carboxylic acid group wherein the —OH of the carboxyl group has been replaced with another substituent. Thus, an acyl group can be represented by RC(═O)—, wherein R is an alkyl, substituted alkyl, aralkyl, aryl or substituted aryl group as defined herein. As such, the term “acyl” specifically includes arylacyl groups, such as an acetylfuran and a phenacyl group. Specific examples of acyl groups include, but are not limited to, acetyl and benzoyl.

The term “amino” refers to the —NH₂, the —NHR, and the —NR₂ groups, wherein R is alkyl, substituted alkyl, aryl, substituted aryl, or aralkyl, as well as to amino functionalities in N-heterocycles (e.g., morpholine, etc).

The term “primary amino” refers to the group —NH₂.

The term “aminoalkyl” refers to —NHR or —NR₂ groups.

The term “hydroxyl” refers to the —OH group.

The term “thiol” refers to the —SH group.

A (═O) group refers to a double-bonded oxygen group.

The terms “guanidine,” “guanidine moiety,” and “guanidino” refer to groups having the structure —NR—C(═NR′)—N(R″)₂, wherein each R, R′, and R″ are independently H, alkyl, substituted alkyl, aralkyl, aryl or substituted aryl, as defined hereinabove. “Unsubstituted guanidine” can refer to the group —NH—C(═NH)—NH₂. “Guanidinium” can refer to an acid salt form of a guanidino group.

The term “amino acid moiety” refers to a substituent formed from (e.g., formed by the abstraction of a hydrogen atom) a molecule comprising both an amino group and a carboxylic acid (i.e., —C(═O)OH) functionality. Amino acid moieties can be formed from any of the natural amino acids (i.e., glycine, proline, alanine, valine, leucine, isoleucine, methionine, cysteine, phenylalanine, tyrosine, tryptophan, histidine, lysine, arginine, glutamine, asparagine, glutamic acid, aspartic acid, serine or threonine) in either the D or L form. Alternatively, the amino acid moiety can be formed from a non-natural amino acid, such as a non-genetically encoded amino acid or a synthetic amino acid.

Representative non-genetically encoded amino acids include but are not limited to 2-aminoadipic acid; 3-aminoadipic acid; β-aminopropionic acid; 2-aminobutyric acid; 4-aminobutyric acid (piperidinic acid); 6-aminocaproic acid; 2-aminoheptanoic acid; 2-aminoisobutyric acid; 3-aminoisobutyric acid; 2-aminopimelic acid; 2,4-diaminobutyric acid; desmosine; 2,2′-diaminopimelic acid; 2,3-diaminopropionic acid; N-ethylglycine; N-ethylasparagine; hydroxylysine; allo-hydroxylysine; 3-hydroxyproline; 4-hydroxyproline; isodesmosine; allo-isoleucine; N-methylglycine (sarcosine); N-methylisoleucine; N-methylvaline; norvaline; norleucine; and ornithine.

The amino acid moiety can also be a derivatized amino acid moiety, for example, wherein the carboxylic acid group has been derivatized to form a carbobenzoxy group, a t-butyloxycarbonyl group, a chloroacetyl group or a formyl groups. Free carboxyl groups can also be derivatized to form salts, methyl and ethyl esters or other types of esters or hydrazides. Free hydroxyl groups can be derivatized to form O-acyl or O-alkyl derivatives. The imidazole nitrogen of histidine can be derivatized to form N-im-benzylhistidine.

The term “peptidyl group” refers to a substituent formed from a polypeptide, i.e., a polymer comprising amino acid residues (i.e., mono- or divalent groups formed from amino acids) covalently attached to one another via amide linkages. The peptidyl group can comprise any combination of natural, non-natural or derivatized amino acid residues. In some embodiments, the peptidyl group can comprises a monovalent polypeptide having between about 2 and about 50 (e.g., 2, 3, 4, 5, 6, 7, 8, 9, 10, 15, 20, 25, 30, 35, 40, 45, and 50) amino acid residues.

When the term “independently selected” is used, the substituents being referred to (e.g., R groups, such as groups R₁ and R₂, or groups X and Y), can be identical or different. For example, both R₁ and R₂ can be substituted alkyls, or R₁ can be hydrogen and R₂ can be a substituted alkyl, and the like.

The term “and/or” when used to describe two or more activities, conditions, or outcomes refers to situations wherein both of the listed conditions are included or wherein only one of the two listed conditions are included.

“Pharmaceutically acceptable” refers to those salts, carriers, compositions, and/or dosage forms that are, within the scope of sound medical judgment, suitable for contact with the tissues of human beings and animals without excessive toxicity, irritation, allergic response, or other problems or complications commensurate with a reasonable benefit/risk ratio. Thus, in some embodiments, the presently disclosed compounds can be provided in formulations comprising the compound and a carrier that is pharmaceutically acceptable for use in humans.

By “in vitro sample” is meant a sample comprising material that is not present in a living subject. For example, the “in vitro sample” can comprise purified SK, an isolated cell, a cell culture, or a cellular or tissue extract comprising SK.

The term “ceramidase-related” can refer to reverse ceramidase activity and to the activities of ceramide/dhceramide synthases and acyl-transferases.

The term “cancer metastasis” refers to the spread of a primary tumor into another organ.

As used herein, the term “effective amount” can refer to a dosage sufficient to provide treatment for the disease state being treated. This can vary depending on the patient, the disease and the treatment being effected. The recognition that the use of an effective amount can be a noticeable change in the severity of a symptom of the disease or disorder as recognized by the subject or by medical or veterinary personnel. In some embodiments, the recognition can relate to a change in the level of a biological marker of the disease or disorder, including the reduction of cellular S1P levels, the elevation of cellular Cer levels, or the shrinkage in the size of a tumor. In some embodiments, the effective amount is about 10 μM.

As used herein, the terms “water-soluble” or “essentially completely water soluble” are meant to define any compound that is soluble in water in an amount of about 15 mg/mL or greater, at about 20° C. In some embodiments, the compound is soluble in water in an amount of about 25 mg/mL or greater at about 37° C. The term “essentially completely water soluble” is meant to include compounds or compositions that are generally water soluble, but which can (for example, as a side product of synthesis) include a small amount of non-water soluble material.

### II. Sphingo-Guanidines

The presently disclosed subject matter provides in some embodiments sphingosine kinase (SK) inhibitors that are analogs of bioactive lipophilic amino alcohols. In some embodiments, the inhibitors combine a sphingolipid based backbone, or an analog thereof, with a guanidine moiety introduced at the sphingolipid C2 position in lieu of an amino group. Without being bound to any one theory, it is believed that the guanidine moiety can generate increased hydrogen bonding interactions with amino acid moieties inside the catalytic site of SK as compared to an amino group. Further, guanidine is believed to be able to interact directly with adenosine triphosphate (ATP) in the catalytic center of some enzymes and to be able to impede (e.g., partially or fully) the phosphorylation reaction. See Onda et al., (1996) J. Am. Chem. Soc., 118, 8524-8530. Thus, the presently disclosed guanidine-sphingosines can act as dual site/dual mode SK inhibitors. In addition, conjugation of sphingosine or a sphingosine analog with a guanidine moiety provides polar lipids. Thus, in some embodiments, the presently disclosed compounds can have improved water solubility over previously reported SK inhibitors, particularly when in a conjugated salt form (e.g., a hydrochloride salt). Fully water soluble sphingosine kinase inhibitors have not been previously reported. See French et al., (2006) J. Pharm. Exp. Ther., 318, 586; and Nageswara et al., (1976) J. Biol. Chem., 251, 6981.

### II.A. Compounds of Formula (I)

In some embodiments, the presently disclosed sphingosine kinase inhibitor is a compound of Formula (I):

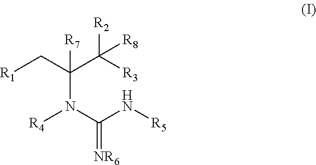

wherein:

R₁ is selected from the group comprising H, alkyl, OH, (═O), (═N—OH), and SH;

R₂ is selected from the group comprising H, alkyl, OH, SH, (═O), and (═N—OH);

R₃ is selected from the group comprising H, alkyl, substituted alkyl, unsaturated alkyl, aryl, substituted aryl, aralkyl, OH, alkoxyl, aryloxyl, aralkoxyl, amino, aminoalkyl, an aminoacid moiety, and a peptidyl moiety;

R₄ and R₅ are independently selected from the group comprising H, C(═NH)—NH₂, acyl, alkyl, substituted alkyl, aralkyl, aryl, and substituted aryl;

R₆ is selected from the group comprising H, CN, alkyl, substituted alkyl, aryl, substituted aryl, aralkyl, and acyl;

R₇ is selected from the group comprising H, alkyl, substituted alkyl, aralkyl, aryl and substituted aryl; and

R₈ is present or absent, and when present is selected from the group comprising H, alkyl, substituted alkyl, aralkyl, aryl and substituted aryl; or a pharmaceutically acceptable salt thereof.

As shown in Scheme 1, the compounds of Formula (I) can include, but are not limited to, N-guanidino-sphingosines, A; N-guanidino-dihydrosphingosines, B; N-guanidino-dehydrosphingosines, C; N-guanidino-phytosphigosines, D; N-guanidino-3-keto-dihydrosphingosines, E; N-guanidino-serine tetradecaneamides, F; 2-N-guanidino-1-phenyl-1,3-propanols, G; 2-N-guanidino-1-phenyl-1-propanols, H; 2-N-guanidino-3-phenyl-1-propanols, I; 2-N-guanidino-1,3-butanols, J; 3-N-guanidino-2-butanols, K; 2-N-guanidino-1-butanols, L; 2-N-guanidino-2-[2-(4-octylphenyl)ethyl]propane-1,3-diols, M; and their R₄, R₅, and R₆ analogs.

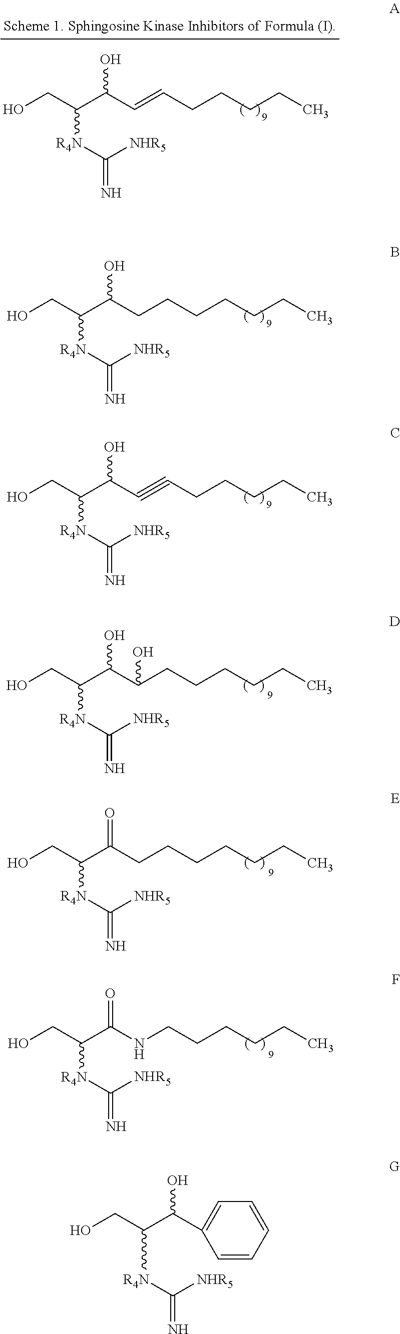

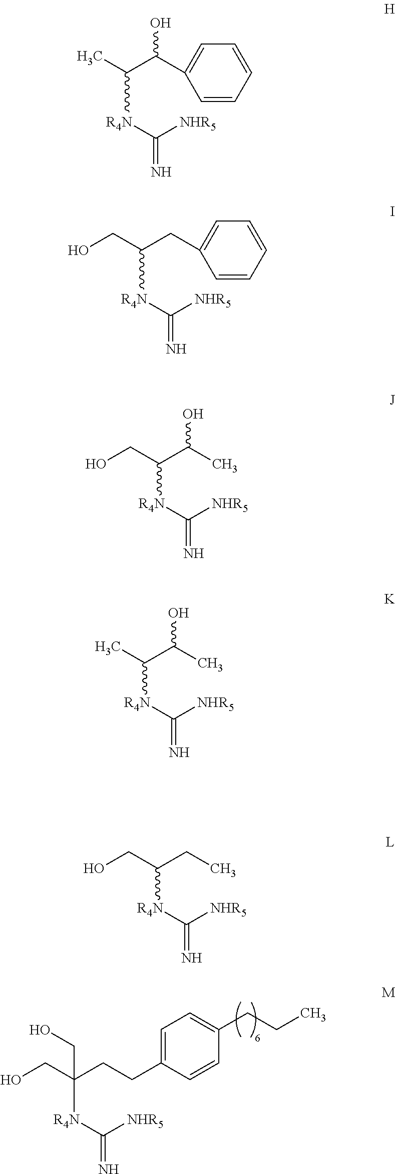

In some embodiments, R₃ is an unsaturated alkyl group, such as an alkenyl or an alkynyl group. In some embodiments, the R₃ alkenyl or alkynyl group can have a structure —CH═CHR or —C≡CR, wherein R is alkyl (e.g., saturated, straight chain, branched or cyclic alkyl). In some embodiments, R₃ can be a C₁₅ alkyl or C₁₅ substituted alkyl group (e.g., C₁₅ saturated alkyl, C₁₅ hydroxy-substituted alkyl, C₁₅ alkenyl, or C₁₅ alkynyl), corresponding to the length of the alkenyl chain in sphingosine, i.e.,:

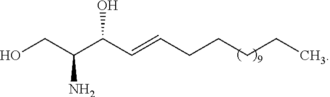

However, R₃ is not limited to C₁₅ alkyl or C₁₅ substituted alkyl groups and, alternatively, can be H, C₁-C₂₀ alkyl, or C₁-C₂₀ substituted (i.e., a C₁, C₂, C₃, C₄, C₅, C₆, C₇, C₈, C₉, C₁₀, C₁₁, C₁₂, C₁₃, C₁₄, C₁₅, C₁₆, C₁₇, C₁₈, C₁₉, or C₂₀ alkyl or substituted alkyl group). In some embodiments, R₃ can be an aminoalkyl group or an aryl group, including, but not limited to phenyl or substituted phenyl. In some embodiments, R₃ can be an aralkyl group. In some embodiments, the R₃ aralkyl group has the structure -alkyl-aryl-alkyl. In some embodiments, R₃ can be hydroxy-substituted alkyl, for example, having the structure —CH(OH)-alkyl.

In some embodiments, R₁ and R₂ are independently selected from H and OH. In some embodiments, R₁ and R₂ are each OH. In some embodiments, R₂ is (═O) and R₈ is absent.

In some embodiments, R₄ is selected from H and C₁-C₆ alkyl. In some embodiments, R₄ and R₅ are each H. In some embodiments, R₆ is H.

In some embodiments, R₇ is H or substituted alkyl. In some embodiments, the substituted alkyl is a hydroxy-substituted alkyl (e.g., —CH₂OH).

In some embodiments, the compound of Formula (I) is selected from D-erythro-2-guanidino-sphingosine, L-erythro-2-guanidino-sphinogosine, or mixtures and/or a salt thereof. Thus, in some embodiments, the compound has a structure (or structures) of the formula:

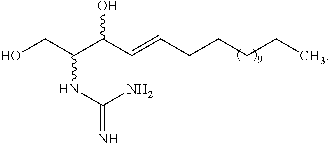

In some embodiments, the compound of Formula (I) is essentially completely water soluble. Thus, in some embodiments, the compound of Formula (I) is essentially completely soluble at a concentration of about 15 mg/mL or greater (e.g., about 15, 16, 17, 18, 19, 20, 21, 22, 23, 24, 25, 26, 27, 28, 29, or 30 mg/mL or greater) at about 20° C. In some embodiments, the compound of Formula (I) is essentially completely water soluble at a concentration of about 25 mg/ml or greater at about 37° C.

### II. B. Pharmaceutally Acceptable Salts

In some embodiments, the compound of Formula (I) is a pharmaceutically acceptable salt. By “pharmaceutically acceptable salt” is meant salts that are acceptable for use in animal subjects, including humans. Thus, in some embodiments, provided is a salt that is pharmaceutically acceptable for use in humans. Such pharmaceutically acceptable salts include, but are not limited to, gluconate, lactate, acetate, dichloroacetate, tartarate, citrate, phosphate, maleate, borate, nitrate, sulfate, and hydrochloride salts.

The salts of the compounds described herein can be prepared, for example, by reacting the free base of a compound of Formula (I) with the desired acid in solution. Suitable acids for forming salts of the compounds of Formula (I) include inorganic acids, such as, but not limited to, hydrochloric acid, hydrobromic acid, nitric acid, phosphoric acid, sulfuric acid, and perchloric acid, and the like, and organic acids, such as, but not limited to, acetic acid, oxalic acid, (D) or (L) malic acid, maleic acid, methanesulfonic acid, ethanesulfonic acid, p-toluenesulfonic acid, salicylic acid, tartaric acid, citric acid, succinic acid, or malonic acid, and the like. Salts can also be formed by reacting a compound of Formula (I) comprising an acidic proton with a compound that can provide an ion to replace the acidic proton, such as, for example, a metal ion, e.g., an alkali metal ion, an alkaline earth metal ion, or an aluminum ion; or with a compound that can coordinate with the acidic proton, e.g., ethanolamine, diethanolamine, triethanolamine, tromethamine, N-methylglucamine, and the like. After the reaction is complete, the salts can be crystallized from solution by the addition of an appropriate amount of solvent in which the salt is insoluble. In some embodiments, the pharmaceutically acceptable salt is a hydrochloride salt.

In some embodiments, the compound of Formula (I) is a compound of Formula (Ia):

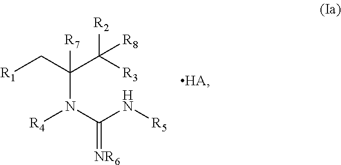

wherein:

R₁ is selected from the group comprising H, alkyl, OH, (═O), (═N—OH), and SH;

R₂ is selected from the group comprising H, alkyl, OH, SH, (═O), and (═N—OH);

R₃ is selected from the group comprising H, alkyl, substituted alkyl, unsaturated alkyl , aryl, substituted aryl, aralkyl, OH, alkoxyl, aryloxyl, aralkoxyl, amino, aminoalkyl, an aminoacid moiety, and a peptidyl moiety;

R₄ and R₅ are independently selected from the group comprising H, C(═NH)—NH₂, acyl, alkyl, substituted alkyl, aralkyl, aryl, and substituted aryl;

R₆ is selected from the group comprising H, CN, alkyl, substituted alkyl, aryl, substituted aryl, aralkyl, and acyl;

R₇ is selected from the group comprising H, alkyl, substituted alkyl, aralkyl, aryl, and substituted aryl;

R₈ is present or absent, and when present is selected from the group comprising H, alkyl, substituted alkyl, aralkyl, aryl and substituted aryl; and

A is a pharmaceutically acceptable inorganic or organic acid.

Thus, in some embodiments, the compound of Formula (la) is a salt of Formula (I) wherein A can be selected from the group including, but not limited to, chloride, bromide, sulfate, acetate, dichloroacetate, benzoate, and tartrate.

### III. Uses of Compounds of Formula (I)

In some embodiments, the presently disclosed subject matter provides a method of inhibiting sphigosine kinase (SK) by contacting a sample with a compound of Formula (I):

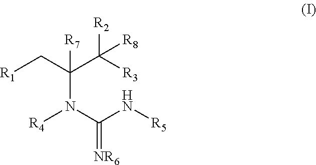

wherein:

R₁ is selected from the group comprising H, alkyl, OH, (═O), (═N—OH), and SH;

R₂ is selected from the group comprising H, alkyl, OH, SH, (═O), and (═N—OH);

R₃ is selected from the group comprising H, alkyl, substituted alkyl, unsaturated alkyl, aryl, substituted aryl, aralkyl, OH, alkoxyl, aryloxyl, aralkoxyl, amino, aminoalkyl, an aminoacid moiety, and a peptidyl moiety;

R₄ and R₅ are independently selected from the group comprising H, C(═NH)—NH₂, acyl, alkyl, substituted alkyl, aralkyl, aryl, and substituted aryl;

R₆ is selected from the group comprising H, CN, alkyl, substituted alkyl, aryl, substituted aryl, aralkyl, and acyl;

R₇ is selected from the group comprising H, alkyl, substituted alkyl, aralkyl, aryl and substituted aryl; and

R₈ is present or absent, and when present is selected from the group comprising H, alkyl, substituted alkyl, aralkyl, aryl and substituted aryl;

or a pharmaceutically acceptable salt thereof. In some embodiments, the compound of Formula (I) is a compound of Formula (Ia).

In some embodiments, the compound selectively inhibits one isoform of SK compared to the other. In some embodiments, the compound selectively inhibits SK1. Selective inhibition can be demonstrated by a compound's having a smaller inhibitory concentration (i.e., a smaller 50% inhibitory concentration (IC₅₀) for one isoform compared to another. In some embodiments, the compound can have an IC₅₀ for SK1 that is at least about 1.5 times (e.g., about 1.5 times, 2.5 times, 5, times, 7.5 times, 10 times, 20 times, 50 times, 100 times) smaller than its IC₅₀for SK2. In some embodiments, the compound can have an IC₅₀ for SK1 that is at least about 5 times smaller than its IC₅₀ for SK2. In some embodiments, the compound can have an IC₅₀ for SK1 that is about 7.5 times smaller than its IC₅₀ for SK2.

In some embodiments, the compound has an IC₅₀ for SK (e.g., SK1) that is smaller than about 1.0 μM (about 0.99, 0.95, 0.90, 0.80, 0.70, 0.60, 0.50, 0.40, 0.30, 0.20, 0.10, 0.05 μM or smaller). In some embodiments, the IC₅₀ is smaller than about 0.40 μM. In some embodiments, the IC₅₀ is less than about 100 nM. In some embodiments, the IC₅₀ is about 40 nM.

In some embodiments, the sample is an in vitro cell sample or an in vivo cell sample. Thus, the sample can include whole cells in culture media or lysed cells containing an SK (e.g., SK1). The sample can also include cells present in a tissue, plasma, or organ sample or present in a subject. Thus, the presently disclosed methods can be used in scientific and/or medical research related to SK activity. In some embodiments, the presently disclosed subject matter provides a method of increasing the level of ceramide in a cell and/or decreasing the level of S1P in a cell, the method comprising contacting the cell with a compound that inhibits SK activity.

Without being bound to any one theory, it is believed that the presently disclosed compounds can, in some embodiments, act as lipid mimics and inhibit the enzymatic function of SK by blocking the SK substrate receptor site and/or by interactions of a guanidinium moiety on the compound with ATP phosphate sub-units, similar to previously reported interactions of arginine with ATP. In particular, arginine moieties in kinases can interact with ATP and can be involved in the phosphoryl-group transfer from ATP to the kinase substrate. Recent NMR studies have confirmed that the guanidinium group of arginine can interact with ATP under physiological conditions. Thus, in some embodiments, the presently disclosed compounds can be used as model molecular probes to study in situ the mechanism of enzymatic phosphorylation of bioactive lipids using NMR techniques. The compounds can also be used, in some embodiments, as molecular probes to study the structure and metabolic function of sphingolipid metabolizing enzymes, and to study the regulatory functions of sphingolipids in vitro.

The presently disclosed subject matter also provides methods involving the use of the compounds of the presently disclosed subject matter for the treatment, prophylaxis, management, or amelioration of one or more symptoms associated with various diseases, disorders and/or other medical conditions related to undesirable SK activity, and/or ceramide and/or S1P levels. For instance, SK is known to be involved in signaling pathways that are abnormally activated in a variety of diseases, including hyperproliferative, inflammatory, and angiogenic diseases. See, for example, U.S. Patent Application Publication No. 2008/0167352 to Smith et al.

Accordingly, in some embodiments, the presently disclosed subject matter provides a method of treating or preventing a disease or disorder associated with undesirable ceramidase, ceramidase-related, and/or sphinogsine kinase activity in a subject, wherein the method comprises administering to the subject in need of treatment an effective amount of a compound of Formula (I):

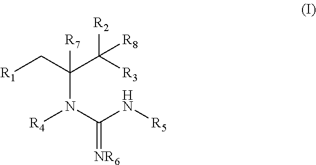

wherein:

R₁ is selected from the group comprising H, alkyl, OH, (═O), (═N—OH), and SH;

R₂ is selected from the group comprising H, alkyl, OH, SH, (═O), and (═N—OH);

R₃ is selected from the group comprising H, alkyl, substituted alkyl, unsaturated alkyl, aryl, substituted aryl, aralkyl, OH, alkoxyl, aryloxyl, aralkoxyl, amino, aminoalkyl, an aminoacid moiety, and a peptidyl moiety;

R₄ and R₅ are independently selected from the group comprising H, C(═NH)—NH₂, acyl, alkyl, substituted alkyl, aralkyl, aryl, and substituted aryl;

R₆ is selected from the group comprising H, CN, alkyl, substituted alkyl, aryl, substituted aryl, aralkyl, and acyl;

R₇ is selected from the group comprising H, alkyl, substituted alkyl, aralkyl, aryl and substituted aryl; and

R₈ is present or absent, and when present is selected from the group comprising H, alkyl, substituted alkyl, aralkyl, aryl and substituted aryl;

or a pharmaceutically acceptable salt thereof. In some embodiments, the compound of Formula (I) is a compound of Formula (Ia).

In some embodiments, the presently disclosed subject matter provides a method of increasing the intracellular levels of a ceramide and/or decreasing intracellular levels of S1P in a subject comprising administering to the subject an effective amount of a compound that inhibits the SK activity of the SK protein in the subject's cells.

In some embodiments, the composition that inhibits SK is administered to a subject therapeutically and/or prophylactically: (1) in diseases or disorders involving an increased (relative to normal or desired) level of SK protein or function, for example, in subjects where SK protein is biologically overactive or overexpressed; or (2) in diseases or disorders wherein in vitro (or in vivo) assays indicate the utility of SK inhibitor administration. The increased level in SK protein or function can be readily detected, e.g., by obtaining a tissue sample from a subject (e.g., from biopsy tissue) and assaying it in vitro for RNA or protein levels, structure and/or activity of the expressed SK RNA or protein. Many methods standard in the art can be thus employed, including but not limited to SK enzyme assays, immunoassays to detect and/or visualize SK protein (e.g., Western blot, immunoprecipitation followed by sodium dodecyl sulfate polyacrylamide gel electrophoresis, immunocytochemistry, etc.) and/or hybridization assays to detect SK expression by detecting and/or visualizing SK mRNA (e.g., Northern assays, dot blots, in situ hybridization, etc.), etc.

In some embodiments, the compositions of the presently disclosed subject matter are used to treat cancer, cancer metastasis, atherosclerosis, stenosis, inflammation, asthma, and atopic dermatitis. In some embodiments, the compositions of the presently disclosed subject matter are used to promote wound healing and/or to treat situations involving abnormal wound healing.

S1P and ceramide have opposing effects on cancer cell proliferation and apoptosis. Sphingomyelin is not only a building block for cellular membranes, but also serves as the precursor for potent lipid messengers that can have cellular effects. Stimulus-induced metabolism of these lipids is critically involved in cancer cell biology.

Ceramide is produced by the hydrolysis of sphingomyelin in response to growth factors or other stimuli. Ceramide induces apoptosis in tumor cells and can enhance apoptosis in response to anti-cancer drugs, but can be further hydrolyzed by the action of ceramidase to produce sphingosine. Sphingosine is then phosphorylated by SK to produce S1P, which is a critical second messenger that exerts proliferative and antiapoptotic actions. Thus, the balance between cellular concentrations of ceramide and S1P can determine whether a cell proliferates or undergoes apoptosis. Upon exposure to mitogens or intracellular oncoproteins, the cells can experience a rapid increase in intracellular levels of S1P and depletion of ceramide levels. This can promote cell survival and proliferation. In contrast, activation of sphingomyelinase in the absence of activation of ceramidase and/or SK can result in the accumulation of ceramide and subsequent apoptosis.

According to the presently disclosed subject matter, disorders involving cell hyperproliferation and/or dysfunctional sphingolipid signal transduction are treated and/or prevented by administration of a composition that inhibits SK function to a subject. These diseases and disorders include, but are not limited to, diseases and disorders related to cell proliferation, cell attachment, cell immigration, granulation tissue development, primary and metastatic neoplastic diseases, inflammation, cardiovascular disease, stroke, ischemia, and/or atherosclerosis. Diseases and disorders involving cell overproliferation that can be treated and/or prevented include, but are not limited to cancers, pre-malignant conditions (e.g., hyperplasia, metaplasia, dysplasia), benign tumors, hyperproliferative disorders, and benign dysproliferative disorders. In some embodiments, the hyperproliferative disorder includes, but is not limited to, psoriasis, a mesangial cell proliferative disorder, atherosclerosis and restenosis. Atherosclerosis and restenosis, for example, can be brought about by hyperproliferation of vascular smooth muscle cells. Psoriasis can be characterized by local keratinocyte hyperproliferation, T-cell-mediated inflammation, and localized angiogenesis. Mesangial cell proliferative disorders are disorders brought about by abnormal hyperproliferation of mesangial cells in the kidney, including various renal diseases, such as, but not limited to, glomerulonephritis, diabetic nephropathy, malignant nephrosclerosis, thrombotic microangiopathy syndromes, transplant rejections, and glomerulopathies.

Cancer is characterized by an increase in the number of abnormal cells derived from a given normal tissue, invasion of adjacent tissues by these abnormal cells, and lymphatic or blood-borne dissemination of the abnormal cells to distant sites in the subject. Malignancies and related disorders, particularly metastatic cancer, which can be treated, prevented, managed, and/or ameliorated by administration of a composition of the presently disclosed subject matter that inhibits SK function are discussed below (for a review of such disorders, see Fishman et al., Medicine, 2^{nd} Ed., J.B. Lippincott Co., Philadelphia, 1985).

In some embodiments, disorders in which cell proliferation is deficient or is desired can be treated or prevented by administration of a composition of the presently disclosed subject matter that promotes SK function to a subject.

The presently disclosed subject matter encompasses methods for treating and/or preventing diseases and disorders wherein the treatment or prevention would be improved by administration of the compounds of the presently disclosed subject matter.

In some embodiments, “treatment” or “treating” refers to an amelioration of disease or disorder, or at least one discernible symptom thereof. “Treatment” or “treating” also refers to an amelioration of at least one measurable physical parameter associated with a disease or disorder that is not necessarily discernible by the subject. “Treatment” or “treating” can also refer to inhibiting the progression of a disease or disorder either physically (e.g., stabilization of a discernible symptom), physiologically (e.g., stabilization of a physical parameter), or both. “Treatment” or “treating” also refers to delaying the onset of a disease or disorder.

In some embodiments, the methods and compositions of the presently disclosed subject matter are useful as a preventative measure against disease or disorder. As used herein, “prevention” or “preventing” refers to a reduction of the risk of acquiring a given disease or disorder.

In some embodiments, the presently disclosed subject matter provides a method of treating cancer comprising administering to a subject in need of treatment thereof an effective amount of a compound of Formula (I) (e.g., a compound of Formula (Ia).

In some embodiments, the presently disclosed subject matter provides methods for treating or preventing diseases or disorders comprising administration of a compound of the presently disclosed subject matter in combination with other treatments.

Cancers and related disorders that can be treated and/or prevented by methods and compositions of the presently disclosed subject matter include, but are not limited to the following: leukemias such as but not limited to, acute leukemia, acute lymphocytic leukemia, acute myelocytic leukemias such as myeloblastic, promyelocytic, myelomonocytic, monocytic, erythroleukemia leukemias and myelodysplastic syndrome, chronic leukemias such as but not limited to, chronic myelocytic (granulocytic) leukemia, chronic lymphocytic leukemia, hairy cell leukemia; polycythemia vera; lymphomas such as but not limited to Hodgkin's disease, non-Hodgkin's disease; multiple myelomas such as but not limited to smoldering multiple myeloma, non-secretory myeloma, osteosclerotic myeloma, plasma cell leukemia, solitary plasmacytoma and extramedullary plasmacytoma; Waldenstrom's acroglobulinemia; monoclonal gammopathy of undetermined significance; benign monoclonal gammopathy; heavy chain disease; bone and connective tissue sarcomas such as but not limited to bone sarcoma, osteosarcoma, chondrosarcoma, Ewing's sarcoma, malignant giant cell tumor, fibrosarcoma of bone, chordoma, periosteal sarcoma, soft-tissue sarcomas, angiosarcoma (hemangiosarcoma), fibrosarcoma, Kaposi's sarcoma, leiomyosarcoma, liposarcoma, lymphangiosarcoma, neurilemmoma, rhabdomyosarcoma, synovial sarcoma; brain tumors such as but not limited to, glioma, astrocytoma, brain stem glioma, ependymoma, oligodendroglioma, non-glial tumor, acoustic neurinoma, craniopharyngioma, medulloblastoma, meningioma, pineocytoma, pineoblastoma, primary brain lymphoma; breast cancer including but not limited to adenocarcinoma, lobular (small cell) carcinoma, intraductal carcinoma, medullary breast cancer, mucinous breast cancer, tubular breast cancer, papillary breast cancer, Paget's disease, and inflammatory breast cancer; adrenal cancer such as but not limited to pheochromocytoma and adrenocortical carcinoma; thyroid cancer such as but not limited to papillary or follicular thyroid cancer, medullary thyroid cancer and anaplastic thyroid cancer; pancreatic cancer such as but not limited to, insulinoma, gastrinoma, glucagonoma, vipoma, somatostatin-secreting tumor, and carcinoid or islet cell tumor; pituitary cancers such as but limited to Cushing's disease, prolactin-secreting tumor, acromegaly, and diabetes insipidus; eye cancers such as but not limited to ocular melanoma such as iris melanoma, choroidal melanoma, and cilliary body melanoma, and retinoblastoma; vaginal cancers such as squamous cell carcinoma, adenocarcinoma, and melanoma; vulvar cancer such as squamous cell carcinoma, melanoma, adenocarcinoma, basal cell carcinoma, sarcoma, and Paget's disease; cervical cancers such as but not limited to, squamous cell carcinoma, and adenocarcinoma; uterine cancers such as but not limited to endometrial carcinoma and uterine sarcoma; ovarian cancers such as but not limited to, ovarian epithelial carcinoma, borderline tumor, germ cell tumor, and stromal tumor; head and neck squamous cell cancers (HNSCCs), esophageal cancers such as but not limited to, squamous cancer, adenocarcinoma, adenoid cyctic carcinoma, mucoepidermoid carcinoma, adenosquamous carcinoma, sarcoma, melanoma, plasmacytoma, verrucous carcinoma, and oat cell (small cell) carcinoma; stomach cancers such as but not limited to, adenocarcinoma, fungating (polypoid), ulcerating, superficial spreading, diffusely spreading, malignant lymphoma, liposarcoma, fibrosarcoma, and carcinosarcoma; colon cancers; rectal cancers; liver cancers such as but not limited to hepatocellular carcinoma and hepatoblastoma, gallbladder cancers such as adenocarcinoma; cholangiocarcinomas such as but not limited to papillary, nodular, and diffuse; lung cancers such as non-small cell lung cancer, squamous cell carcinoma (epidermoid carcinoma), adenocarcinoma, large-cell carcinoma and small-cell lung cancer; testicular cancers such as but not limited to germinal tumor, seminoma, anaplastic, classic (typical), spermatocytic, non-seminoma, embryonal carcinoma, teratoma carcinoma, choriocarcinoma (yolk-sac tumor), prostate cancers such as but not limited to, adenocarcinoma, leiomyosarcoma, and rhabdomyosarcoma; penal cancers; oral cancers such as but not limited to squamous cell carcinoma;

basal cancers; salivary gland cancers such as but not limited to adenocarcinoma, mucoepidermoid carcinoma, and adenoidcystic carcinoma; pharynx cancers such as but not limited to squamous cell cancer, and verrucous; skin cancers such as but not limited to, basal cell carcinoma, squamous cell carcinoma and melanoma, superficial spreading melanoma, nodular melanoma, lentigo malignant melanoma, acral lentiginous melanoma; kidney cancers such as but not limited to renal cell cancer, adenocarcinoma, hypernephroma, fibrosarcoma, transitional cell cancer (renal pelvis and/or uterer); Wilms' tumor; bladder cancers such as but not limited to transitional cell carcinoma, squamous cell cancer, adenocarcinoma, carcinosarcoma. In addition, cancers include myxosarcoma, osteogenic sarcoma, endotheliosarcoma, lymphangioendotheliosarcoma, mesothelioma, synovioma, hemangioblastoma, epithelial carcinoma, cystadenocarcinoma, bronchogenic carcinoma, sweat gland carcinoma, sebaceous gland carcinoma, papillary carcinoma and papillary adenocarcinomas. For a review of such disorders, see Fishman et al., Medicine, 2^{nd} Ed., J.B. Lippinocott Co., Philadelphia, 1985; and Murphy et al., Informed Decisions: The Complete Book of Cancer Diagnosis, Treatment, and Recovery, Viking Penguin, Penguin Books, U.S.A., Inc., New York, 1997).

In some embodiments, the methods and compositions of the presently disclosed subject matter are used for the treatment and/or prevention of breast cancer, prostate cancer, melanoma, and/or non small cell lung cancer.

The compositions of the presently disclosed subject matter that inhibit SK activity can also be administered to treat pre-malignant conditions and/or to prevent progression of a pre-malignant condition to a neoplastic or malignant state. Such prophylactic or therapeutic use is indicated in conditions known or suspected of preceding progression to neoplasia or cancer, in particular, where non-neoplastic cell growth comprising hyperplasia, metaplasia, or most particularly, dysplasia has occurred. For review of such abnormal growth conditions, see Robbins and Angell, Basic Pathology, 2^{nd} Ed., W.B. Saunders Co., Philadelphia, 1976, pages 68-79).

Alternatively or in addition to the presence of abnormal cell growth characterized as hyperplasia, metaplasia, or dysplasia, the presence of one or more characteristics of a transformed phenotype or of a malignant phenotype displayed in vivo or displayed in vitro by a cell sample from a subject can indicate the desirability of prophylactic and/or therapeutic administration of a composition that inhibits SK function. Characteristics of a transformed phenotype can include morphology changes, looser substratum attachment, loss of contact inhibition, loss of anchorage dependence, protease release, increased sugar transport, decreased serum requirement, expression of fetal antigens, etc.

In some embodiments, Ieukoplakia, a benign-appearing hyperplastic or dysplastic lesion of the epithelium, or Bowen's disease, a carcinoma in situ, are pre-neoplastic lesions indicative of the desirability of prophylactic intervention. In some embodiments, fibrocystic disease (cystic hyperplasia, mammary dysplasia, particularly adenosis (benign epithelial hyperplasia) is indicative of the desirability of prophylactic intervention.

In other embodiments, a subject which exhibits one or more of the following predisposing factors for malignancy is treated by administration of an effective amount of a compound of the presently disclosed subject matter: a chromosomal translocation associated with a malignancy (e.g., the Philadelphia chromosome for chronic myelogenous leukemia, t(14;18) for follicular lymphoma, etc.), familial polyposis or Gardner's syndrome (possible forerunners of colon cancer), benign monoclonal gammopathy (a possible forerunner of multiple myeloma), and a first degree kinship with persons having a cancer or precancerous disease showing a Mendelian (genetic) inheritance pattern (e.g., familial polyposis of the colon, Gardner's syndrome, hereditary exostosis, polyendocrine adenomatosis, medullary thyroid carcinoma with amyloid production and pheochromocytoma, Peutz-Jeghers syndrome, neurofibromatosis of Von Recklinghausen, retinoblastoma, carotid body tumor, cutaneous melanocarcinoma, intraocular melanocarcinoma, xeroderma pigmentosum, ataxia telangiectasia, Chediak-Higashi syndrome, albinism, Fanconi's aplastic anemia, and Bloom's syndrome; see Robbins and Angell, Basic Pathology, 2^{nd} Ed., W.B. Saunders Co., Philadelphia, 1976, pages 112-113.

The presently disclosed subject matter also encompasses methods for treating and/or preventing a cancer or metastasis in a subject comprising in any order the steps of administering to the subject a compound of Formula (I). In some embodiments, the compound of Formula (I) is a compound of Formula (Ia). In some embodiments, the compositions and methods of the presently disclosed subject matter can be used to prevent, inhibit, and/or reduce the growth and/or metastasis of cancerous cells. The administration of compound inhibits or reduces the growth and/or metastasis of cancerous cells by in some embodiments at least 99%, in some embodiments at least 95%, in some embodiments at least 90%, in some embodiments at least 85%, in some embodiments at least 80%, in some embodiments at least 75%, in some embodiments at least 70%, in some embodiments at least 65%, in some embodiments at least 60%, in some embodiments at least 55%, in some embodiments at least 50%, in some embodiments at least 45%, in some embodiments at least 40%, in some embodiments at least 35%, in some embodiments at least 30%, in some embodiments at least 25%, in some embodiments at least 20%, in some embodiments at least 15%, in some embodiments at least 10%, and in some embodiments at least 5% relative to the growth or metastasis in absence of the administration of said compound.

The presently disclosed subject matter also encompasses methods of disease treatment and/or prevention that provide better therapeutic profiles than current single agent therapies or even current combination therapies. Encompassed by the presently disclosed subject matter are combination therapies that have additive potency or an additive therapeutic effect while reducing or avoiding unwanted or adverse effects.

Other cancer treatment that can be used in combination of the administration of the compounds of the presently disclosed subject matter include the use of one or more compositions which include, but are not limited to, chemoagents, immunotherapeutics, cancer vaccines, anti-angiogenic agents, cytokines, hormone therapies, gene therapies, biological therapies, and radiotherapies. While maintaining and/or enhancing efficacy of treatment, the methods of the presently disclosed subject matter can also increase subject compliance, improve therapy, and/or reduce unwanted or adverse effects.

In some embodiments, a compound of the presently disclosed subject matter is administered to a subject receiving a treatment modality for the treatment of cancer wherein the subject might experience unwanted or adverse effects to treatment with the treatment modality alone (e.g., the treatment modality might be toxic or harmful at its effective dose, administered alone). Given the presently disclosed subject matter, the compound can improve the therapeutic benefit of the treatment modality such that the dosage and/or frequency of administration of the treatment modality can be lowered when administered in conjunction with the compound. In some embodiments, a compound of the presently disclosed subject matter is administered to allow lower and/or less frequent doses of chemotherapy and/or radiation therapy.

In some embodiments, the methods of the presently disclosed subject matter encompass the administration of one or more angiogenesis inhibitors such as, but not limited to angiostatin (plasminogen fragment); anti-angiogenic anti-thrombin III; ANGIOZYME™; ABT-627; Bay 12-9566; Benefin; Bevacizumab; BMS-275291; cartilage-derived inhibitor (CDI); CAI; CD59 complement fragment; CEP-7055; Col 3; Combretastatin A-4; ENDOSTATIN™ (collagen XVIII fragment); Fibronectin fragment; Gro-beta; Halofuginone; Heparinases; Heparin hexasaccharide fragment; HMV833; Human chorionic gonadotropin (hCG); IM-862; Interferon alpha/beta/gamma; Interferon inducible protein (IP-10); Interleukin-12; Kringle 5 (plasminogen fragment); Marimastat; Metalloproteinase inhibitors (TIMPs); 2-Methoxyestradiol; MMI 270 (CGS 27023A); MoAb IMC-1C11; Neovastat; NM-3; Panzem; PI-88; Placental ribonuclease inhibitor; Plasminogen activator inhibitor; Platelet factor-4 (PF4); Prinomastat; Prolactin 16 kDa fragment; Proliferin-related protein (PRP); PTK 787/ZK 222594; Retinoids; Solimastat; Squalamine; SS 3304; SU 5416; SU6668; SU11248; Tetrahydrocortisol-S; tetrathiomolybdate; thalidomide; Thrombospondin-1 (TSP-1); TNP-470; Transforming growth factor-beta (TGF-b); Vasculostatin; Vasostatin (calreticulin fragment); ZD6126; ZD 6474; farnesyl transferase inhibitors (FTI); and bisphosphonates.

Additional examples of anti-cancer agents (e.g., chemotherapeutic) that can be used in conjunction with the presently disclosed subject matter, including pharmaceutical compositions and dosage forms and kits of the presently disclosed subject matter, include, but are not limited to: acivicin; aclarubicin; acodazole hydrochloride; acronine; adozelesin; aldesleukin; altretamine; ambomycin; ametantrone acetate; aminoglutethimide; amsacrine; anastrozole; anthramycin; asparaginase; asperlin; azacitidine; azetepa; azotomycin; batimastat; benzodepa; bicalutamide; bisantrene hydrochloride; bisnafide dimesylate; bizelesin; bleomycin sulfate; brequinar sodium; bropirimine; busulfan; cactinomycin; calusterone; caracemide; carbetimer; carboplatin; carmustine; carubicin hydrochloride; carzelesin; cedefingol; chlorambucil; cirolemycin; cisplatin; cladribine; crisnatol mesylate; cyclophosphamide; cytarabine; dacarbazine; dactinomycin; daunorubicin hydrochloride; decitabine; dexormaplatin; dezaguanine; dezaguanine mesylate; diaziquone; docetaxel; doxorubicin; doxorubicin hydrochloride; droloxifene; droloxifene citrate; dromostanolone propionate; duazomycin; edatrexate; eflornithine hydrochloride; elsamitrucin; enloplatin; enpromate; epipropidine; epirubicin hydrochloride; erbulozole; esorubicin hydrochloride; estramustine; estramustine phosphate sodium; etanidazole; etoposide; etoposide phosphate; etoprine; fadrozole hydrochloride; fazarabine; fenretinide; floxuridine; fludarabine phosphate; fluorouracil; flurocitabine; fosquidone; fostriecin sodium; gemcitabine; gemcitabine hydrochloride; hydroxyurea; idarubicin hydrochloride; ifosfamide; ilmofosine; interleukin II (including recombinant interleukin-2, or rIL-2), interferon alfa-2a; interferon α-2b; interferon α-n1; interferon α-n3; interferon β-I a; interferon γ-I b; iproplatin; irinotecan hydrochloride; lanreotide acetate; letrozole; leuprolide acetate; liarozole hydrochloride; lometrexol sodium; lomustine; losoxantrone hydrochloride; masoprocol; maytansine; mechlorethamine hydrochloride; megestrol acetate; melengestrol acetate; melphalan; menogaril; mercaptopurine; methotrexate; methotrexate sodium; metoprine; meturedepa; mitindomide; mitocarcin; mitocromin; mitogillin; mitomalcin; mitomycin; mitosper; mitotane; mitoxantrone hydrochloride; mycophenolic acid; nocodazole; nogalamycin; ormaplatin; oxisuran; pegaspargase; peliomycin; pentamustine; peplomycin sulfate; perfosfamide; pipobroman; piposulfan; piroxantrone hydrochloride; plicamycin; plomestane; porfimer sodium; porfiromycin; prednimustine; procarbazine hydrochloride; puromycin; puromycin hydrochloride; pyrazofurin; riboprine; rogletimide; safingol; safingol hydrochloride; semustine; simtrazene; sparfosate sodium; sparsomycin; spirogermanium hydrochloride; spiromustine; spiroplatin; streptonigrin; streptozocin; sulofenur; talisomycin; tecogalan sodium; tegafur; teloxantrone hydrochloride; temoporfin; teniposide; teroxirone; testolactone; thiamiprine; thioguanine; thiotepa; tiazofurin; tirapazamine; toremifene citrate; trestolone acetate; triciribine phosphate; trimetrexate; trimetrexate glucuronate; triptorelin; tubulozole hydrochloride; uracil mustard; uredepa; vapreotide; verteporfin; vinblastine sulfate; vincristine sulfate; vindesine; vindesine sulfate; vinepidine sulfate; vinglycinate sulfate; vinleurosine sulfate; vinorelbine tartrate; vinrosidine sulfate; vinzolidine sulfate; vorozole; zeniplatin; zinostatin; zorubicin hydrochloride. Other anti cancer drugs include, but are not limited to: 20-epi-1,25 dihydroxyvitamin D3; 5-ethynyluracil; abiraterone; aclarubicin; acylfulvene; adecypenol; adozelesin; aldesleukin; ALL-TK antagonists; altretamine; ambamustine; amidox; amifostine; aminolevulinic acid; amrubicin; amsacrine; anagrelide; anastrozole; andrographolide; angiogenesis inhibitors; antagonist D; antagonist G; antarelix; anti-dorsalizing morphogenetic protein-1; antiandrogen, prostatic carcinoma; antiestrogen; antineoplaston; antisense oligonucleotides; aphidicolin glycinate; apoptosis gene modulators; apoptosis regulators; apurinic acid; ara-CDP-DL-PTBA; arginine deaminase; asulacrine; atamestane; atrimustine; axinastatin 1; axinastatin 2; axinastatin 3; azasetron; azatoxin; azatyrosine; baccatin III derivatives; balanol; batimastat; BCR/ABL antagonists; benzochlorins; benzoylstaurosporine; beta lactam derivatives; beta-alethine; betaclamycin B; betulinic, acid; bFGF inhibitor; bicalutamide; bisantrene; bisaziridinylspermine; bisnafide; bistratene A; bizelesin; breflate; bropirimine; budotitane; buthionine sulfoximine; calcipotriol; calphostin C; camptothecin derivatives; canarypox IL-2; capecitabine; carboxamide amino triazole; carboxyamidotriazole; CaRest M3; CARN 700; cartilage derived inhibitor; carzelesin; casein kinase inhibitors (ICOS); castanospermine; cecropin B; cetrorelix; chlorins; chloroquinoxaline sulfonamide; cicaprost; cis porphyrin; cladribine; clomifene analogues; clotrimazole; collismycin A; collismycin B; combretastatin A4; combretastatin analogue; conagenin; crambescidin 816; crisnatol; cryptophycin 8; cryptophycin A derivatives; curacin A; cyclopentanthraquinones; cycloplatam; cypemycin; cytarabine ocfosfate; cytolytic factor; cytostatin; dacliximab; decitabine; dehydrodidemnin B; deslorelin; dexamethasone; dexifosfamide; dexrazoxane; dexverapamil; diaziquone; didemnin B; didox; diethylnorspermine; dihydro-5-azacytidine; dihydrotaxol, 9-; dioxamycin; diphenyl spiromustine; docetaxel; docosanol; dolasetron; doxifluridine; droloxifene; dronabinol; duocarmycin SA; ebselen; ecomustine; edelfosine; edrecolomab; eflornithine; elemene; emitefur; epirubicin; epristeride; estramustine analogue; estrogen agonists; estrogen antagonists; etanidazole; etoposide phosphate; exemestane; fadrozole; fazarabine; fenretinide; filgrastim; finasteride; flavopiridol; flezelastine; fluasterone; fludarabine; fluorodaunorunicin hydrochloride; forfenimex; formestane; fostriecin; fotemustine; gadolinium texaphyrin; gallium nitrate; galocitabine; ganirelix; gelatinase inhibitors; gemcitabine; glutathione inhibitors; hepsulfam; heregulin; hexamethylene bisacetamide; hypericin; ibandronic acid; idarubicin; idoxifene; idramantone; ilmofosine; ilomastat; imidazoacridones; imiquimod; immunostimulant peptides; insulin like growth factor 1 receptor inhibitor; interferon agonists; interferons; interleukins; iobenguane; iododoxorubicin; ipomeanol, 4-; iroplact; irsogladine; isobengazole; isohomohalicondrin B; itasetron; jasplakinolide; kahalalide F; lamellarin-N triacetate; lanreotide; leinamycin; lenograstim; lentinan sulfate; leptolstatin; letrozole; leukemia inhibiting factor; leukocyte alpha interferon; leuprolide+estrogen+progesterone; leuprorelin; levamisole; liarozole; linear polyamine analogue; lipophilic disaccharide peptide; lipophilic platinum compounds; lissoclinamide 7; lobaplatin; lombricine; lometrexol; lonidamine; losoxantrone; lovastatin; loxoribine; lurtotecan; lutetium texaphyrin; lysofylline; lytic peptides; maitansine; mannostatin A; marimastat; masoprocol; maspin; matrilysin inhibitors; matrix metalloproteinase inhibitors; menogaril; merbarone; meterelin; methioninase; metoclopramide; MIF inhibitor; mifepristone; miltefosine; mirimostim; mismatched double stranded RNA; mitoguazone; mitolactol; mitomycin analogues; mitonafide; mitotoxin fibroblast growth factor saporin; mitoxantrone; mofarotene; molgramostim; monoclonal antibody, human chorionic gonadotrophin; monophosphoryl lipid A+myobacterium cell wall sk; mopidamol; multiple drug resistance gene inhibitor; multiple tumor suppressor 1-based therapy; mustard anticancer agent; mycaperoxide B; mycobacterial cell wall extract; myriaporone; N-acetyldinaline; N-substituted benzamides; nafarelin; nagrestip; naloxone+pentazocine; napavin; naphterpin; nartograstim; nedaplatin; nemorubicin; neridronic acid; neutral endopeptidase; nilutamide; nisamycin; nitric oxide modulators; nitroxide antioxidant; nitrullyn; O6-benzylg uanine; octreotide; okicenone; oligonucleotides; onapristone; ondansetron; ondansetron; oracin; oral cytokine inducer; ormaplatin; osaterone; oxaliplatin; oxaunomycin; paclitaxel; paclitaxel analogues; paclitaxel derivatives; palauamine; palmitoylrhizoxin; pamidronic acid; panaxytriol; panomifene; parabactin; pazelliptine; pegaspargase; peldesine; pentosan polysulfate sodium; pentostatin; pentrozole; perflubron; perfosfamide; perillyl alcohol; phenazinomycin; phenylacetate; phosphatase inhibitors; picibanil; pilocarpine hydrochloride; pirarubicin; piritrexim; placetin A; placetin B; plasminogen activator inhibitor; platinum complex; platinum compounds; platinum-triamine complex; porfimer sodium; porfiromycin; prednisone; propyl bis-acridone; prostaglandin J2; proteasome inhibitors; protein A-based immune modulator; protein kinase C inhibitor; protein kinase C inhibitors, microalgal; protein tyrosine phosphatase inhibitors; purine nucleoside phosphorylase inhibitors; purpurins; pyrazoloacridine; pyridoxylated hemoglobin polyoxyethylene conjugate; raf antagonists; raltitrexed; ramosetron; ras farnesyl protein transferase inhibitors; ras inhibitors; ras GAP inhibitor; retelliptine demethylated; rhenium Re 186 etidronate; rhizoxin; ribozymes; RII retinamide; rogletimide; rohitukine; romurtide; roquinimex; rubiginone B1; ruboxyl; safingol; saintopin; SarCNU; sarcophytol A; sargramostim; Sdi 1 mimetics; semustine; senescence derived inhibitor 1; sense oligonucleotides; signal transduction inhibitors; signal transduction modulators; single chain antigen binding protein; sizofiran; sobuzoxane; sodium borocaptate; sodium phenylacetate; solverol; somatomedin binding protein; sonermin; sparfosic acid; spicamycin D; spiromustine; splenopentin; spongistatin 1; squalamine; stem cell inhibitor; stem-cell division inhibitors; stipiamide; stromelysin inhibitors; sulfinosine; superactive vasoactive intestinal peptide antagonist; suradista; suramin; swainsonine; synthetic glycosaminoglycans; tallimustine; tamoxifen methiodide; tauromustine; tazarotene; tecogalan sodium; tegafur; tellurapyrylium; telomerase inhibitors; temoporfin; temozolomide; teniposide; tetrachlorodecaoxide; tetrazomine; thaliblastine; thiocoraline; thrombopoietin; thrombopoietin mimetic; thymalfasin; thymopoietin receptor agonist; thymotrinan; thyroid stimulating hormone; tin ethyl etiopurpurin; tirapazamine; titanocene bichloride; topsentin; toremifene; totipotent stem cell factor; translation inhibitors; tretinoin; triacetyluridine; triciribine; trimetrexate; triptorelin; tropisetron; turosteride; tyrosine kinase inhibitors; tyrphostins; UBC inhibitors; ubenimex; urogenital sinus-derived growth inhibitory factor; urokinase receptor antagonists; vapreotide; variolin B; vector system, erythrocyte gene therapy; velaresol; veramine; verdins; verteporfin; vinorelbine; vinxaltine; vitaxin; vorozole; zanoterone; zeniplatin; zilascorb; and zinostatin stimalamer. Representative additional anti-cancer drugs are 5-fluorouracil and leucovorin. These two agents are particularly useful when used in methods employing thalidomide and a topoisomerase inhibitor.

In some embodiments, the treatment of the presently disclosed subject matter further includes the administration of one or more immunotherapeutic agents, such as antibodies and immunomodulators, which include, but are not limited to, HERCEPTIN®, RITUXAN®, OVAREX™, PANOREX®, BEC2, IMC-C225, VITAXIN™, CAMPATH® I/H, Smart MI95, LYMPHOCIDE™, Smart I D10, and ONCOLYM™, rituximab, gemtuzumab, or trastuzumab.

In some embodiments, the treatment of the presently disclosed subject matter further includes administering one or more anti-angiogenic agents, which include, but are not limited to, angiostatin, thalidomide, kringle 5, endostatin, other Serpins, anti-thrombin, 29 kDa N-terminal and 40 kDa C-terminal proteolytic fragments of fibronectin, 16 kDa proteolytic fragment of prolactin, 7.8 kDa proteolytic fragment of platelet factor-4, a 13-amino acid peptide corresponding to a fragment of platelet factor-4 (see Maione et al., Cancer Res., 51, 2077-2083, (1991)), a 14-amino acid peptide corresponding to a fragment of collagen I (see Tolsma et al., J. Cell Biol., 122, 497-511 (1993)), a 19 amino acid peptide corresponding to a fragment of Thrombospondin I (see Tolsma et al., J. Cell Biol., 122, 497-511 (1993)), a 20-amino acid peptide corresponding to a fragment of SPARC (see Sage et al., J. Cell Biochem., 57, 127-140 (1995)), or any fragments, family members, or derivatives thereof, including pharmaceutically acceptable salts thereof.

In some embodiments, the treatment method further comprises the use of radiation.

In some embodiments, the treatment method further comprises the administration of one or more cytokines, which include, but are not limited to, lymphokines, tumor necrosis factors, tumor necrosis factor-like cytokines, lymphotoxin-α, lymphotoxin-β, interferon-α, interferon-β, macrophage inflammatory proteins, granulocyte monocyte colony stimulating factor, interleukins (including, but not limited to, interleukin-1, interleukin-2, interleukin-6, interleukin-12, interleukin-15, interleukin-18), OX40, CD27, CD30, CD40, or CD137 ligands, Fas/Fas ligand, 4-1BBL, endothelial monocyte activating protein or any fragments, family members, or derivatives thereof, including pharmaceutically acceptable salts thereof.

In some embodiments, the treatment method further comprises hormonal treatment. Hormonal therapeutic treatments comprise hormonal agonists, hormonal antagonists (e.g., flutamide, tamoxifen, leuprolide acetate (LUPRON™), LH-RH antagonists), inhibitors of hormone biosynthesis and processing, steroids (e.g., dexamethasone, retinoids, betamethasone, cortisol, cortisone, prednisone, dehydrotestosterone, glucocorticoids, mineralocorticoids, estrogen, testosterone, progestins), antigestagens (e.g., mifepristone, onapristone), and anti-androgens (e.g., cyproterone acetate).

Other disorders of proliferation that can benefit from inhibition of SK include cardiovascular diseases.

Vascular interventions, including angioplasty, stenting, atherectomy, and grafting for the treatment of cardiovascular diseases are often complicated by undesirable effects. One of the adverse reactions to vascular intervention include endothelial and smooth muscle cell proliferation which can lead to hyperplasia, or more specifically, restenosis which is the re-clogging of the artery, occlusion of blood vessels, reperfusion injury, platelet aggregation, and calcification. In this model, an injurious stimulus induces expression of growth-stimulatory cytokines such as interleukin 1 and tumor necrosis factor. See Libby et al., Circulation, 86, 11147-11152 (1992). There is evidence which shows that ceramide inhibits the growth of endothelia and smooth muscle cells of the coronary artery.

Various therapies have been attempted to treat or prevent stenosis or restenosis. However, there remains a need for therapies directed to the prevention and treatment of cardiovascular diseases caused by hyperplasia of endothelia and smooth muscle cells. Since it has been shown that ceramide inhibits the growth of endothelia and smooth muscle cells of the coronary artery, it therefore can be desirable to raise the level of ceramide for the treatment and prevention of cardiovascular diseases. Recently, Kester and co-workers showed that ceramide used in angioplasty prevents restenosis. See Kester et al., Circ. Res., 87, 282-288 (2000). Alternative, and more effectively, one aspect of the presently disclosed subject matter provides treatment and prevention of restenosis by adjusting the level of ceramide through administering a compound of the presently disclosed subject matter.

Accordingly, it therefore can be desirable to raise the level of ceramide for the treatment and prevention of cardiovascular diseases. This can be accomplished by adjusting the intracellular level of ceramide by using the compositions and methods of the presently disclosed subject matter. The outcome of a treatment is to at least produce in a treated subject a healthful benefit, which in the case of cardiovascular diseases, includes, but is not limited to a reduced risk of re-clogging of arteries after a vascular intervention procedure and improved circulation.

S1P has several effects on cells that mediate immune functions. For example, platelets, monocytes, and mast cells secrete S1P upon activation, promoting inflammatory cascades. It is believed that SK activation is required for the related signaling responses. In addition, deregulation of apoptosis in phagocytes can be an important component of chronic inflammatory diseases. S1P has been found to protect neutrophils and macrophages in response to inflammatory stresses, such as TNFa. Additional information regarding the role of S1P and SK in various specific inflammatory and/or autoimmune conditions can be found in U.S. Patent Application Publication No. 2008/0167352. Accordingly, inhibition of SK (which can reduce levels of S1P) can prevent the hyperproliferation of immune cells that are important for inflammation.

Thus, in some embodiments, the presently disclosed subject matter provides a method for preventing, treating, managing, and/or ameliorating an autoimmune or inflammatory disorder or one or more symptoms thereof, said method comprising administering to a subject in need thereof a prophylactically and/or therapeutically effective amount of a compound of the presently disclosed subject matter and a prophylactically or therapeutically effective amount of one or more immunomodulatory agents.

Examples of autoimmune disorders include, but are not limited to, alopecia areata, ankylosing spondylitis, antiphospholipid syndrome, autoimmune Addison's disease, autoimmune diseases of the adrenal gland, autoimmune hemolytic anemia, autoimmune hepatitis, autoimmune oophoritis and orchitis, autoimmune thrombocytopenia, Behcet's disease, bullous pemphigoid, cardiomyopathy, celiac sprue-dermatitis, chronic fatigue immune dysfunction syndrome (CFIDS), chronic inflammatory demyelinating polyneuropathy, Churg-Strauss syndrome, cicatrical pemphigoid, CREST syndrome, cold agglutinin disease, Crohn's disease, discoid lupus, essential mixed cryoglobulinemia, fibromyalgia-fibromyositis, glomerulonephritis, Graves' disease, Guillain-Barre, Hashimoto's thyroiditis, idiopathic pulmonary fibrosis, idiopathic thrombocytopenia purpura (ITP), IgA neuropathy, juvenile arthritis, lichen planus, lupus erthematosus, Ménière's disease, mixed connective tissue disease, multiple sclerosis, type 1 or immune-mediated diabetes mellitus, myasthenia gravis, pemphigus vulgaris, pernicious anemia, polyarteritis nodosa, polychrondritis, polyglandular syndromes, polymyalgia rheumatica, polymyositis and dermatomyositis, primary agammaglobulinemia, primary biliary cirrhosis, psoriasis, psoriatic arthritis, Raynauld's phenomenon, Reiter's syndrome, Rheumatoid arthritis, sarcoidosis, scleroderma, Sjogren's syndrome, stiff-man syndrome, systemic lupus erythematosus, lupus erythematosus, takayasu arteritis, temporal arteristis/ giant cell arteritis, ulcerative colitis, uveitis, vasculitides such as dermatitis herpetiformis vasculitis, vitiligo, and Wegener's granulomatosis. Examples of inflammatory disorders include, but are not limited to, asthma, encephilitis, inflammatory bowel disease (IBD), chronic obstructive pulmonary disease (COPD), allergic disorders, septic shock, pulmonary fibrosis, undifferentitated spondyloarthropathy, undifferentiated arthropathy, arthritis, inflammatory osteolysis, and chronic inflammation resulting from chronic viral or bacteria infections. Some autoimmune disorders are associated with an inflammatory condition. Thus, there is overlap between what is considered an autoimmune disorder and an inflammatory disorder. Therefore, some autoimmune disorders can also be characterized as inflammatory disorders.

The presently disclosed subject matter provides methods of preventing, treating, managing, and/or ameliorating an autoimmune or inflammatory disorder and/or one or more symptoms thereof, said methods comprising administering to a subject in need thereof a compound of the presently disclosed subject matter and one or more immunomodulatory agents. The immunomodulatory agents are administered to a subject with an autoimmune and/or inflammatory disorder whose mean absolute lymphocyte count is in some embodiments less than 500 cells/mm³, in some embodiments less than 550 cells/mm³, in some embodiments less than 600 cells/mm³, in some embodiments less than 650 cells/mm³, in some embodiments less than 700 cells/mm³, in some embodiments less than 750 cells/mm³, in some embodiments less than 800 cells/mm³, in some embodiments less than 850 cells/mm³ and in some embodiments less than 900 cells/mm³. Thus, in some embodiments, prior to or subsequent to the administration of one or more dosages of one or more immunomodulatory agents to a subject with an autoimmune or inflammatory disorder, the absolute lymphocyte count of said subject is determined by techniques well-known to one of skill in the art, including, e.g., flow cytometry or trypan blue counts.

Examples of immunomodulatory agents include, but are not limited to, methotrexate, leflunomide, cyclophosphamide, cyclosporine A, and macrolide antibiotics (e.g., FK506 (tacrolimus)), methylprednisolone (MP), corticosteroids, steroids, mycophenolate mofetil, rapamycin (sirolim us), mizoribine, deoxyspergualin, brequinar, malononitriloamindes (e.g., leflunamide), T cell receptor modulators, and cytokine receptor modulators. Examples of T cell receptor modulators include, but are not limited to, anti-T cell receptor antibodies (e.g., anti-CD4 monoclonal antibodies, anti-CD3 monoclonal antibodies, anti-CD8 monoclonal antibodies, anti-CD40 ligand monoclonal antibodies, anti-CD2 monoclonal antibodies) and CTLA4-immunoglobulin. Examples of cytokine receptor modulators include, but are not limited to, soluble cytokine receptors (e.g., the extracellular domain of a TNF-α receptor or a fragment thereof, the extracellular domain of an IL-1β receptor or a fragment thereof, and the extracellular domain of an IL-6 receptor or a fragment thereof), cytokines or fragments thereof (e.g., interleukin (IL)-2, IL-3, IL-4, IL-5, IL-6, IL-7, IL-8, IL-9, IL-10, IL-11, IL-12, IL-15, TNF-α, TNF-β, interferon (IFN)-α, IFN-β, IFN-γ, and GM-CSF), anti-cytokine receptor antibodies (e.g., anti-IL-2 receptor antibodies, anti-IL-4 receptor antibodies, anti-IL-6 receptor antibodies, anti-IL-10 receptor antibodies, and anti-IL-12 receptor antibodies), anti-cytokine antibodies (e.g., anti-IFN receptor antibodies, anti-TNF-α antibodies, anti-IL-1β antibodies, anti-IL-6 antibodies, and anti-IL-12 antibodies).

Any anti-inflammatory agent well-known to one of skill in the art can be used in the compositions and methods of the presently disclosed subject matter. Non-limiting examples of anti-inflammatory agents include non-steroidal anti-inflammatory drugs (NSAIDs), steroidal anti-inflammatory drugs, beta-agonists, anticholingeric agents, and methyl xanthines. Examples of NSAIDs include, but are not limited to, aspirin, ibuprofen, celecoxib (CELEBREX™), diclofenac (VOLTAREN™), etodolac (LODINE™), fenoprofen (NALFON™), indomethacin (INDOCIN™), ketoralac (TORADOL™), oxaprozin (DAYPRO™), nabumentone (RELAFEN™), sulindac (CLINORIL™), tolmentin (TOLECTIN™), rofecoxib (VIOXX™), naproxen (ALEVE™, NAPROSYN™) ketoprofen (ACTRON™) and nabumetone (RELAFEN™). Such NSAIDs function by inhibiting a cyclooxgenase enzyme (e.g., COX-1 and/or COX-2). Examples of steroidal anti-inflammatory drugs include, but are not limited to, glucocorticoids, dexamethasone (DECADRON™), cortisone, hydrocortisone, prednisone (DELTASONE™), prednisolone, triamcinolone, azulfidine, and eicosanoids such as prostaglandins, thromboxanes, and leukotrienes.

In some embodiments, the presently disclosed subject matter relates to the treatment of diseases or disorders that involve undesired angiogenesis. Angiogenesis refers to a state in the body wherein various growth factors or other stimuli promote the formation of new blood vessels. Angiogenesis plays a role in a variety of diseases, including, but not limited to, diabetic retinopathy, arthritis, cancer, psoriasis, Kaposi's sarcoma, hemangiomas, myocardial angiogenesis, atherscelortic plaque neovascularization, and ocular angiogenic diseases such as choroidal neovascularization, retinopathy of prematurity (retrolental fibroplasias), macular degeneration, corneal graft rejection, rubeosis, neuroscular glacoma and Oster Webber syndrome. Generally, excessive angiogenesis allows the progression of the disease and/or produces undesired effects. S1P has been shown to stimulate NFKB production, which can lead to production of COX-2, adhesion molecules and vascular endothelial growth factor (VEGF), all of which have been linked to angiogenesis. Thus, inhibiting SK, the key enzyme responsible for S1P production, can lead to a reduction in angiogenesis.

The presently disclosed subject matter also relates to the treatment of disorders involving deficient cell proliferation (growth) and/or in which cell proliferation is otherwise desired (e.g., degenerative disorders, growth deficiencies, lesions, physical trauma) by administering compounds that agonize, (promote) SK function. Other disorders that can benefit from activation of SK are neurodegenerative disorders (e.g., Alzheimer's disease), and disorders of aging such as immune dysfunction.

As discussed above, like treatment of neoplastic conditions, successful treatment of cardiovascular diseases, inflammation, or the above-mentioned diseases can be brought about by techniques which serve to decrease SK activity.

General techniques that can be employed for the determination of effective doses and administration of such compounds are known to the skilled artisan. Any technique which serves to selectively administer chemicals to a cell population of interest can be used, for example, by using a delivery complex. Such a delivery complex can comprise an appropriate chemical and a targeting agent. Such targeting agents can comprise, for example, sterols, lipids, viruses or target cell specific binding agents.

### IV. Pharmaceutical Preparation and Methods of Administration

The compounds described herein can be administered to a subject at therapeutically effective doses to treat or prevent diseases and disorder discussed above. A therapeutically effective dose refers to that amount of a compound sufficient to result in a healthful benefit in the treated subject. See the Physicians' Desk Reference® (53^{rd} ed., 1999).

The term “subject” as used herein refers to a member of any invertebrate or vertebrate species. The methods of the presently disclosed subject matter are particularly useful for warm-blooded vertebrates. Thus, the presently disclosed subject matter concerns mammals and birds. More particularly provided is the treatment of mammals such as humans, as well as those mammals of importance due to being endangered (such as Siberian tigers), of economic importance (animals raised on farms for consumption by humans) and/or social importance (animals kept as pets or in zoos) to humans, for instance, carnivores other than humans (such as cats and dogs), swine (pigs, hogs, and wild boars), ruminants (such as cattle, oxen, sheep, giraffes, deer, goats, bison, and camels), and horses. Also provided is the use of the disclosed methods and compositions on birds, including those kinds of birds that are endangered, kept in zoos or as pets (e.g., parrots), as well as fowl, and more particularly domesticated fowl, e.g., poultry, such as turkeys, chickens, ducks, geese, guinea fowl, and the like, as they are also of economic importance to humans.

Thus, the subject to which a compound of the presently disclosed subject matter is administered is in some embodiments an animal, including but not limited to a mammal such as a non-primate (e.g., cows, pigs, horses, chickens, cats, dogs, rats, etc.), or a primate (e.g., a monkey such as acynomolgous monkey or a human). In some embodiments, the subject is a human. The composition of the presently disclosed subject matter can be utilized for the prevention of a variety of cancers, e.g., in individuals who are predisposed as a result of familial history or in individuals with an enhanced risk to cancer due to environmental factors (e.g., exposure to cancer-causing chemicals).

The methods and compositions of the presently disclosed subject matter can be used in subjects who are treatment naïve and/or in subjects who have previously received and/or are currently receiving treatment with other pharmaceutical agents or combinations, including but not limited to anti-cancer agents. Other subjects can include subjects that have metastasis or no metastasis.

The methods and compositions of the presently disclosed subject matter are useful not only in untreated subjects but are also useful in the treatment of subjects partially or completely un-responsive to other treatments. In some embodiments, the presently disclosed subject matter provides methods and compositions useful for the treatment of diseases or disorders in subjects that have been shown to be or might be refractory or non-responsive to therapies comprising the administration of other agents.

Suitable subjects for treatment according to the presently disclosed methods and/or with the presently disclosed compounds can be chosen by finding an increased level of SK activity in the subject. For example, suitable subjects can be chosen based on an absence or decreased level of ceramide and/or by an increased level of S1P in a biological sample taken from the subject. An absence or decreased level in ceramide level or function (or increased level of S1P) can be readily detected, e.g., by obtaining a tissue sample (e.g., from biopsy tissue) and assaying it in vitro for ceramide (or SIP).

### IV.A. Effective Dose

Toxicity and therapeutic efficacy of compositions can be determined by standard pharmaceutical procedures in cell cultures or experimental animals (e.g., for determining the LD₅₀ (the dose lethal to 50% of the population) and the ED₅₀ (the dose therapeutically effective in 50% of the population)). The dose ratio between toxic and therapeutic effects is the therapeutic index and it can be expressed as the ratio LD₅₀/ED₅₀. In some embodiments, compounds that exhibit large therapeutic indices are employed. While compounds that exhibit toxic side effects can be used, care should be taken to design a delivery system that targets such compounds to the site of affected tissue in order to minimize potential damage to uninfected cells and, thereby, reduce side effects.

The data obtained from the cell culture assays and animal studies can be used in formulating a range of dosage for use in humans. The dosage of such compounds lies in some embodiments within a range of circulating concentrations that include the ED₅₀ with little or no toxicity. For example, the dosage can range from in some embodiments 10 nM to 100 μM (e.g., about 10 nM, 50 nM, 100 nM, 250 nM, 500 nM, 750 nM, 1.0 μM, 5 μM, 10 μM, 25 μM, 50 μM, 75 μM, or 100 μM) and in some embodiments 1 to 10 μM or greater. The dosage can vary within this range depending upon the dosage form employed and the route of administration utilized. For any composition used in the methods of the presently disclosed subject matter, the therapeutically effective dose can be estimated initially from cell culture assays. A dose can be formulated in animal models to achieve a circulating plasma concentration range that includes the IC₅₀ (i.e., the concentration of the test compound which achieves a half-maximal inhibition of symptoms) as determined in cell culture. Such information can be used to more accurately determine useful doses in humans. Levels in plasma can be measured, for example, by high performance liquid chromatography.

Suitable daily doses for the treatment or prevention of a disorder described herein can be readily determined by those skilled in the art. A recommended dose of a composition of the presently disclosed subject matter is from about 0.1 mg to about 100 mg per day, given as a single once-a-day dose in the morning or as divided doses throughout the day. In some embodiments, a daily dose is from about 2 mg to about 25 mg per day and in some embodiments a daily dose is from about 5 mg to about 10 mg per day.

The anti-cancer activity of the methods and compositions used in accordance with the presently disclosed subject matter also can be determined by using various experimental animal models of such as cancer animal models such as scid mouse model or nude mice with human tumor grafts known in the art and described in Yamanaka et al. (Microbiol. Immunol., 45, 507-514 (2001)).

In some embodiments, the methods and compositions of the presently disclosed subject matter are tested in vitro, and then in vivo, for the desired therapeutic or prophylactic activity, prior to use in a subject (e.g., a human). For example, in vitro assays which can be used to determine whether administration of a specific therapeutic protocol is indicated, include in vitro cell culture assays in which a subject tissue sample is grown in culture, and exposed to or otherwise administered a protocol, and the effect of such protocol upon the tissue sample is observed. A lower level of proliferation or survival of the contacted cells indicates that the composition is effective to treat the condition in the subject. Alternatively, instead of culturing cells from a subject, compositions can be screened using cells of a tumor or malignant cell line. Many assays standard in the art can be used to assess such survival and/or growth; for example, cell proliferation can be assayed by measuring ³H-thymidine incorporation, by direct cell count, by detecting changes in transcriptional activity of known genes such as proto-oncogenes (e.g., fos, myc) or cell cycle markers; cell viability can be assessed by trypan blue staining, differentiation can be assessed visually based on changes in morphology, etc.

Prior to testing in humans, compositions for use in the presently disclosed methods can be tested in suitable animal model systems, including but not limited to in rats, mice, chicken, cows, monkeys, rabbits, etc. The principle animal models for cancer known in the art and widely used include mice, as described in Hann et al. (Curr. Opin, Cell Biol., 13, 778-784 (2001)), which is incorporated herein by reference in its entirety.

Further, any assays known to those skilled in the art can be used to evaluate the prophylactic and/or therapeutic utility of the methods and compositions disclosed herein for treatment, prophylaxis, management, and/or amelioration of one or more symptoms associated with a disease or disorder as described herein.

Efficacy in treating inflammatory disorders can be demonstrated by detecting the ability of the compounds of the presently disclosed subject matter or a composition of the presently disclosed subject matter to reduce or inhibit inflammation in an animal and/or to ameliorate or alleviate one or more symptoms associated with an inflammatory disorder. The treatment is considered therapeutic if there is, for example, a reduction is in inflammation and/or amelioration of one or more symptoms following administration of the compound or composition of the presently disclosed subject matter.

### IV.B. Formulations and Use

Various methods can be used to administer a compound of the presently disclosed subject matter. Methods of introduction include but are not limited to intradermal, intramuscular, intraperitoneal, intravenous, subcutaneous, intranasal, epidural, inhalation, insufflation (either through the mouth or the nose), oral, buccal, or rectal routes. The compositions can be administered by any convenient route, for example by infusion or bolus injection, by absorption through epithelial or mucocutaneous linings (e.g., oral mucosa, rectal and intestinal mucosa, etc.) and can be administered together with other biologically active agents. Administration can be systemic or local. In addition, it can be desirable to introduce the pharmaceutical compositions of the presently disclosed subject matter into the central nervous system by any suitable route, including intraventricular and intrathecal injection; intraventricular injection can be facilitated by an intraventricular catheter, for example, attached to a reservoir, such as an Ommaya reservoir. Pulmonary administration can also be employed, e.g., by use of an inhaler or nebulizer and formulation with an aerosolizing agent.

In some embodiments, it is desirable to administer the compositions of the presently disclosed subject matter locally to the area in need of treatment. This can be achieved, for example, and not by way of limitation, by local infusion during surgery, by a catheter, by a suppository, or by an implant. The implant can be a porous, non-porous, or gelatinous material, including membranes, such as sialastic membranes, or fibers, by way of example and not limitation.

In some embodiments, the presently disclosed compounds can be delivered in a vesicle, in particular a liposome. See Langer, Science, 249, 1527-1533 (1990); and Treat et al., in Liposomes in the Therapy of Infectious Disease and Cancer, Lopez-Berestein and Fidler (eds.), Liss, New York, 1989, pp. 317-327 and 353-365.

In some embodiments, the presently disclosed compounds can be delivered in a controlled release system. In some embodiments, a pump is used. See Langer, Science, 249, 1527-1533 (1990); Sefton, CRC Crit. Ref., Biomed. Eng., 14, 201 (1987); Buchwald et al., Surgery, 88, 507 (1980); and Saudek et al., N. Engl. J. Med., 321, 574 (1989). In some embodiments, polymeric materials can be used. See Langer and Wise (eds.), Medical Applications of Controlled Release, CRC Press, Boca Raton, Fla., 1974; Smolen and Ball (eds.), Controlled Drug Bioavailability, Drug Product Design and Performance, Wiley, N.Y., 1984; Ranger and Peppas, J. Macromol. Sci. Rev. Macromol. Chem., 23, 61 (1983); see also Levy et al., Science, 228, 190 (1985); During et al., Ann. Neurol., 25, 351 (1989); and Howard et al., J. Neurosurg., 71, 105 (1989). In some embodiments, a controlled release system can be placed in proximity of the therapeutic target, thus requiring only a fraction of the systemic dose. See Goodson, in Medical Applications of Controlled Release, Langer and Wise (eds.), CRC Press, Boca Raton, Fla., 1974. Other controlled release systems are discussed in Langer (Science, 249, 1527-1533 (1990)).

Other methods of delivery of the therapeutics of the presently disclosed subject matter can be used for example, as described in U.S. Pat. No. 5,679,350, which is incorporated by reference in its entirety.

The presently disclosed subject matter also provides pharmaceutical compositions. Such compositions comprise a therapeutically effective amount of one or more compounds of Formula (I) (e.g., one or more compounds of Formula (Ia)) of the presently disclosed subject matter and a pharmaceutically acceptable carrier. “Pharmaceutically acceptable” can refer to those carriers, compositions, and/or dosage forms that are, within the scope of sound medical judgment, suitable for contact with the tissues of human beings and/or other animals without excessive toxicity, irritation, allergic response, or other problems or complications commensurate with a reasonable benefit/risk ratio. Thus, in some embodiments, the presently disclosed compounds can be provided in formulations comprising the compound and a carrier that is pharmaceutically acceptable for use in humans. In some embodiments, the term “pharmaceutically acceptable” means approved by a regulatory agency of the Federal or a state government or listed in the U.S. Pharmacopeia or other generally recognized pharmacopeia for use in animals, and more particularly in humans.

The term “carrier” refers to a diluent, adjuvant, excipient, or vehicle with which the therapeutic is administered. Such pharmaceutical carriers can be sterile liquids, such as water and oils, including those of petroleum, animal, vegetable or synthetic origin, such as peanut oil, soybean oil, mineral oil, sesame oil and the like. Water is an exemplary carrier when the pharmaceutical composition is administered intravenously. Saline solutions and aqueous dextrose and glycerol solutions can also be employed as liquid carriers, particularly for injectable solutions. Suitable pharmaceutical excipients include starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol, and the like. The composition, if desired, can also contain minor amounts of wetting or emulsifying agents, or pH buffering agents. These compositions can take the form of solutions, suspensions, emulsion, tablets, pills, capsules, powders, sustained-release formulations, and the like. The composition can be formulated as a suppository, with traditional binders and carriers such as triglycerides. Oral formulation can include standard carriers such as pharmaceutical grades of mannitol, lactose, starch, magnesium stearate, sodium saccharine, cellulose, magnesium carbonate, etc. Examples of suitable pharmaceutical carriers are described in Remington's Pharmaceutical Sciences (1990). Such compositions will contain in some embodiments a therapeutically effective amount of the ACDase inhibitor or related prodrug in purified form, together with a suitable amount of carrier so as to provide the form for proper administration to the subject. The formulation should suit the mode of administration.

In some embodiments, the composition is formulated in accordance with routine procedures as a pharmaceutical composition adapted for intravenous administration to human beings. Typically, compositions for intravenous administration are solutions in sterile isotonic aqueous buffer. Where necessary, the composition can also include a solubilizing agent and a local anesthetic such as lidocaine to ease pain at the site of the injection. Generally, the ingredients are supplied either separately or mixed together in unit dosage form, for example, as a dry lyophilized powder or water free concentrate in a hermetically sealed container such as an ampoule or sachette indicating the quantity of active agent. Where the composition is to be administered by infusion, it can be dispensed with an infusion bottle containing sterile pharmaceutical grade water or saline. Where the composition is administered by injection, an ampoule of sterile water for injection or saline can be provided so that the ingredients can be mixed prior to administration.

The compounds of the presently disclosed subject matter can be formulated as neutral or salt forms. Pharmaceutically acceptable salts include those formed with free amino groups such as those derived from hydrochloric, phosphoric, acetic, oxalic, tartaric acids, etc., and those formed with free carboxyl groups such as those derived from sodium, potassium, ammonium, calcium, ferric hydroxides, isopropylamine, triethylamine, 2-ethylamino ethanol, histidine, procaine, etc.

The amounts of the compounds of the presently disclosed subject matter which are effective in the treatment of a particular disorder or condition can depend on the nature of the disorder or condition and can be determined by standard clinical techniques. In addition, in vitro assays and animal models can optionally be employed to help identify optimal dosage ranges. The precise dose to be employed in the formulation can also depend on the route of administration and the seriousness of the disease or disorder, and should be determined according to the judgment of the practitioner and each subject's circumstances.

In some embodiments, the compounds of the presently disclosed subject matter are administered intramuscularly. Suitable dosage ranges for the intramuscular administration are generally in some embodiments about 10 μg to 1 mg per dose and in some embodiments about 10 μg to 100 μg per dose. In some embodiments, the composition is administered in two doses, for example, where the second dose is administered several hours or days (e.g., 24 hours) after the first dose. In some embodiments, a composition of the presently disclosed subject matter is administered in three doses, for example, with one dose being administered on each of days 1, 4, and 7 of a 7-day regimen.

Suppositories generally contain active ingredient in the range of 0.5% to 10% by weight; oral formulations generally contain 10% to 95% active ingredient.

The presently disclosed subject matter also provides a pack or kit for therapeutic use comprising one or more containers filled with one or more of the ingredients of the pharmaceutical compositions of the presently disclosed subject matter. Optionally associated with such container(s) can be a notice in the form prescribed by a governmental agency regulating the manufacture, use, or sale of pharmaceuticals and/or diagnostic products, which notice reflects approval by the agency of manufacture, use, or sale for human administration.

Pharmaceutical compositions for use in accordance with the presently disclosed subject matter can be formulated in conventional manner using one or more physiologically acceptable carriers or excipients.

For oral administration, the pharmaceutical compositions can take the form of, for example, tablets or capsules prepared by conventional means with pharmaceutically acceptable excipients such as binding agents (e.g., pre-gelatinized maize starch, polyvinylpyrrolidone or hydroxypropyl methylcellulose); fillers (e.g., lactose, microcrystalline cellulose or calcium hydrogen phosphate); lubricants (e.g., magnesium stearate, talc or silica); disintegrants (e.g., potato starch or sodium starch glycolate); or wetting agents (e.g., sodium lauryl sulfate). The tablets can be coated by methods well known in the art. Liquid preparations for oral administration can take the form of, for example, solutions, syrups or suspensions, or they can be presented as a dry product for constitution with water or other suitable vehicle before use. Such liquid preparations can be prepared by conventional approaches with pharmaceutically acceptable additives such as suspending agents (e.g., sorbitol syrup, cellulose derivatives or hydrogenated edible fats); emulsifying agents (e.g., lecithin or acacia); non-aqueous vehicles (e.g., almond oil, oily esters, ethyl alcohol or fractionated vegetable oils); and preservatives (e.g., methyl or propyl-p-hydroxybenzoates or sorbic acid). The preparations can also contain buffer salts, flavoring, coloring, and sweetening agents as appropriate.

Preparations for oral administration can be suitably formulated to give controlled release of the active agent in the composition.

For buccal administration, the compositions can take the form of tablets or lozenges formulated in conventional manner.

For administration by inhalation, the compositions for use according to the presently disclosed subject matter can be delivered in the form of an aerosol spray presentation from pressurized packs or a nebulizer, with the use of a suitable propellant (e.g., dichlorodifluoromethane, trichlorofluoromethane, dichlorotetrafluoroethane, carbon dioxide, or other suitable gas). In the case of a pressurized aerosol, the dosage unit can be determined by providing a valve to deliver a metered amount. Capsules and cartridges of e.g., gelatin, for use in an inhaler or insufflator can be formulated containing a powder mix of the composition and a suitable powder base such as lactose or starch.

The compositions can be formulated for parenteral administration (i.e., intravenous or intramuscular) by injection via, for example, bolus injection or continuous infusion. Formulations for injection can be presented in unit dosage form, e.g., in ampoules or in multi-dose containers, with an added preservative. The compositions can take such forms as suspensions, solutions, or emulsions in oily or aqueous vehicles, and can contain formulatory agents such as suspending, stabilizing, and/or dispersing agents. Alternatively, the active ingredient can be in powder form for constitution with a suitable vehicle, e.g., sterile pyrogen-free water, before use.

The compositions can also be formulated in rectal compositions such as suppositories or retention enemas, e.g., containing conventional suppository bases such as cocoa butter or other glycerides.

In addition to the formulations described previously, the compositions can also be formulated as a depot preparation. Such long acting formulations can be administered by implantation (for example subcutaneously or intramuscularly) or by intramuscular injection. Thus, for example, the compositions can be formulated with suitable polymeric or hydrophobic materials (for example, as an emulsion in an acceptable oil) or ion exchange resins, or as sparingly soluble derivatives, for example, as a sparingly soluble salt.

### EXAMPLES

The following Examples provide illustrative embodiments. In light of the present disclosure and the general level of skill in the art, those of skill can appreciate that the following Examples are intended to be exemplary only and that numerous changes, modifications, and alterations can be employed without departing from the scope of the presently disclosed subject matter.

### Example 1

### Preparation of Guanidino-Sphingosine Analogs

Guanidino compounds can be prepared as illustrated in Scheme 2 below, which shows the synthesis of LCL146 and LCL351. Additional guanidino compounds can be prepared similarly using other sphingoid bases and congeners thereof that contain an available primary and secondary amino group available for guanidinylation.

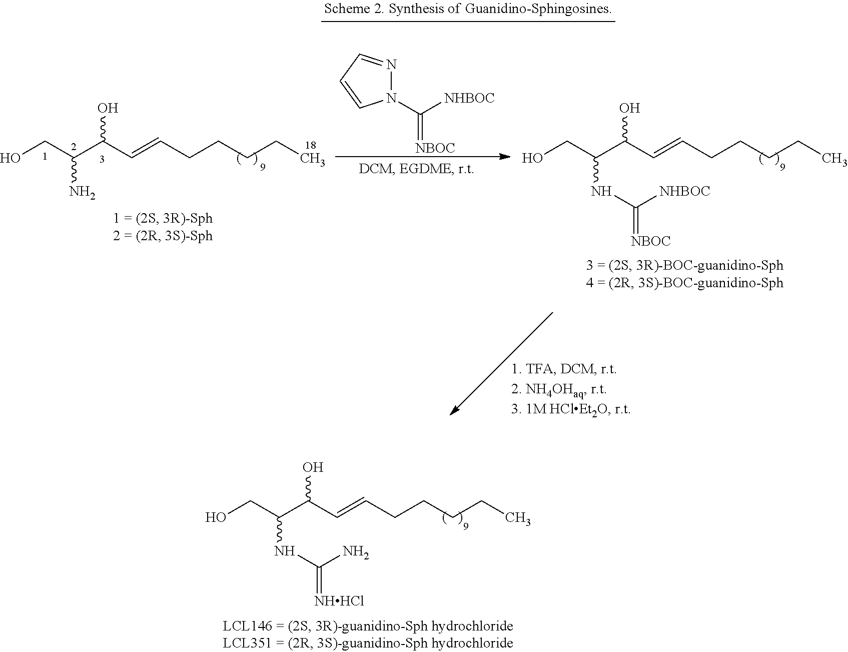

As illustrated in Scheme 2, the condensation of D- or L-erythro-sphingosine (1 or 2) with N,N′-bis(tert-butoxycarbonyl)-1H-pyrazole-1-carboxamidine is performed in dichloromethane (DCM)-dimethylethylene glycol ether (EGDME) solution at room temperature (r.t.), providing 2-N-[N′,N″-bis-(tert-butoxycarbonyl)-1 -carboxamidino]-sphingosine 3 or 4, respectively. Treatment of 3 or 4 with trifluoroacetic acid (TFA) in dry DCM, followed by purification of the crude product by flash column chromatography under basic conditions provided the free base of LCL146 or LCL351, respectively. The compounds were transformed to hydrochloride salts by dissolution in ice-cold ethyl acetate to which was added 1M HCl in diethyl ether at room temperature.

D-erythro-2-N-[1′-[N′,N″-bis-(tert-butoxycarbonyl)]carboxamidino]-sphingosine (3): A mixture of D-erythro-sphingosine (1, 220 mg, 0.74 mmol) and N,N′-bis-(tert-butoxycarbonyl)-1H-pyrazole-1-carboxamidine (98%, 250 mg, 0.79 mmol) in dry DCM-EGDME (5.0 mL, 3:2, v/v) solution was stirred with the exclusion of moisture for 3 hours at room temperature. The solvent was removed under reduced pressure and the residue was purified by flash column chromatography (CHCl₃:MeOH, 50:1, v/v) to give pure 3 was a white solid (338 mg, 85% yield). An analytical sample of 3 was obtained by crystallization from cold anhydrous ethyl acetate-hexane (1:4, v/v, −5° C.) as a white powder, mp: 92-94° C., R_{f}=0.37 (CHCl₃:MeOH, 50:1, v/v); [α]²⁴_{D}=−3.6 and [α]²⁴₃₆₅=−27.6 (c=1.0, MeOH).

L-erythro-2-N-[1′-[N′,N″-bis-(tert-butoxycarbonyl)]carboxamidino]-sphingosine (4): Prepared from L-erythro-sphingosine (2) as described for the synthesis of 3 in 90% yield. Analytical sample of 4 was obtained by crystallization from cold anhydrous ethyl acetate-hexane (1:4, v/v. −5° C.) as a white powder. [α]²⁴_{D}=+3.1 and [α]²⁴₃₆₅=+25.2 (c=0.5, MeOH). Remaining data identical to that for 3.

D-erythro-2-N-(1′-carboxamidino)-sphingosine hydrochloride (i.e., D-e-2-guanidino-sphingosine hydrochloride; LCL146): A mixture of 3 (274 mg, 0.5 mmol) and TFA (3 mL) in dry DCM (2 mL) was stirred for 4 hours at room temperature and then refrigerated overnight. The reaction mixture was evaporated under reduced pressure to dryness and the resultant residue further dried under high vacuum (˜1 torr) for an additional 6 hours at room temperature. The crude product was purified by flash column chromatography (CHCl₃:MeOH, conc. NH₄OH, 70:30:3, v/v) to give the free base of LCL146 as a white amorphous solid (130 mg, 75% yield). This material was dissolved in ice-cold ethyl acetate and a 1M solution of HCl in diethyl ether (1.5 mL) was added dropwise at room temperature. The mixture was evaporated under reduced pressure to dryness and the resulting residue dried in a high vacuum for 2 hours at room temperature to give crude LCL146 as a white solid. An analytical sample of LCL146 was obtained by crystallization from anhydrous cold ethyl acetate-acetone (2:3, v/v, +4° C.) as a white powder (111 mg, 59% yield). TLC R_{f} 0.39 (CHCl₃/CH₃OH/conc. NH₄OH; 70:30:3; v/v); mp: moisture appears at 95° C. and slowly melts >148° C. with decomposition; [α]²⁴_{D}=−16.5 and [α]²⁴₃₆₅=−58.2 (c=1, MeOH); ¹H NMR (500 mHz, MeOD) δ 5.77 (dtd, 1H, J=15.0, 6.7, 0.8, 5-H), 5.47 (ddt, 1H, J=15.0, 7.3, 1.0, 4-H), 4.17 (t, 1H, J=6.2, 3-H), 3.72 (dd, 1H, J=11.1, 3.9, 1-Ha), 3.62 (dd, 1H, J=11.1, 6.9, 1-Hb), 3.55 (m, 1H, 2-H), 2.08 (q, 2H, J=6.9, 6-H), 1.4 (m, 2H, 7-H), 1.27 (m, 20H, CH₂), 0.89 (t, 3H, J=7.0, CH₃); ESI-MS (CH₃OH, relative intensity, %) m/z 342.2 ([MH]⁺, 100), 324.2 ([MH—H₂O]⁺, 62), 294.1(8). Calcd. for [C₁₉H₄₀N₃O₂]⁺ m/z 342.31.

L-erythro-2-N-(1′-carboxamidino)-sphingosine hydrochloride (i. a, L-e-2-guanidino-sphingosine hydrochloride; LCL351): Prepared from 4 as described above for the synthesis of LDL146 in 61% yield. An analytical sample of LCL351 was obtained by crystallization from cold anhydrous ethyl acetate-acetone (2:3, v/v, +4° C.) as a white powder. [α]²⁴_{D}=+14.5 and [α]²⁴₃₅₅=+56.8 (c=1, MeOH). Remaining data was the same as that reported for LCL146.

### Example 2

### Inhibition of Sphingoskine Kinases

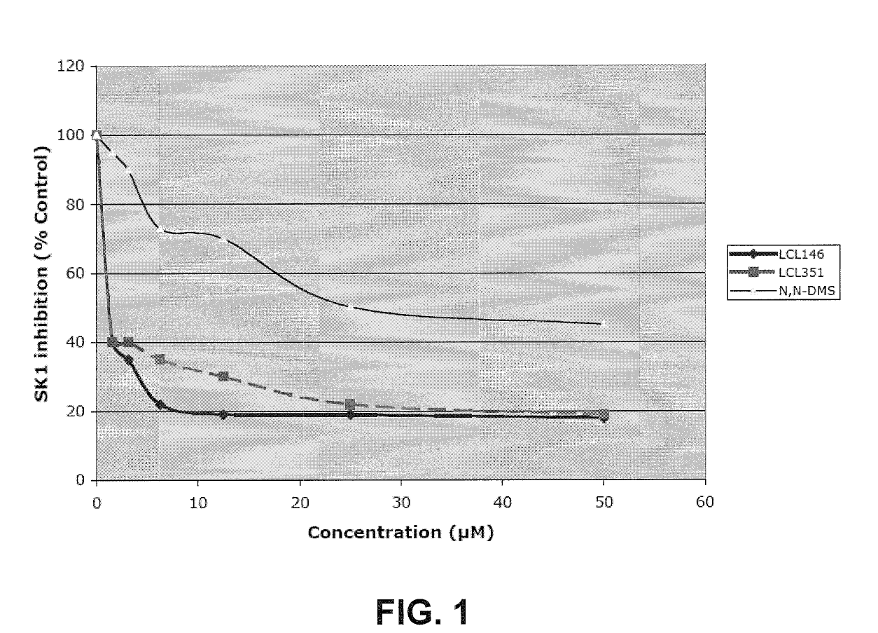

The inhibitory activity of LCL146 and LCL351 for sphingosine kinase 1 (SKI) and sphingosine kinase 2 (SK2) were determined using purified recombinant enzyme. The inhibitory effects of LCL146 and LCL351 on recombinant SK1 activity are also shown in FIG. 1, compared to the inhibitory effects of D-erythro-N,N-dimethyl-sphingosine (N, N-DMS). The 50% inhibitory concentration (IC₅₀) values of SK1 and SK2 inhibition are shown in Table 1, below. The IC₅₀ of LCL351 on SK1 was established as 40 nM. At a low concentration LCL351 acted as a specific inhibitor of SK1.

TABLE 1
In Vitro IC₅₀s of Guanidino-Sphingosines on Sphingosine Kinases.

| | Sphingosine Kinase Isoform | |
|---|---|---|
| Inhibitor | Sphingosine Kinase 1 | Sphingosine Kinase 2 |
| LCL351 | 0.04 μM | 0.30 μM |
| LCL146 | 0.42 μM | 0.74 μM |

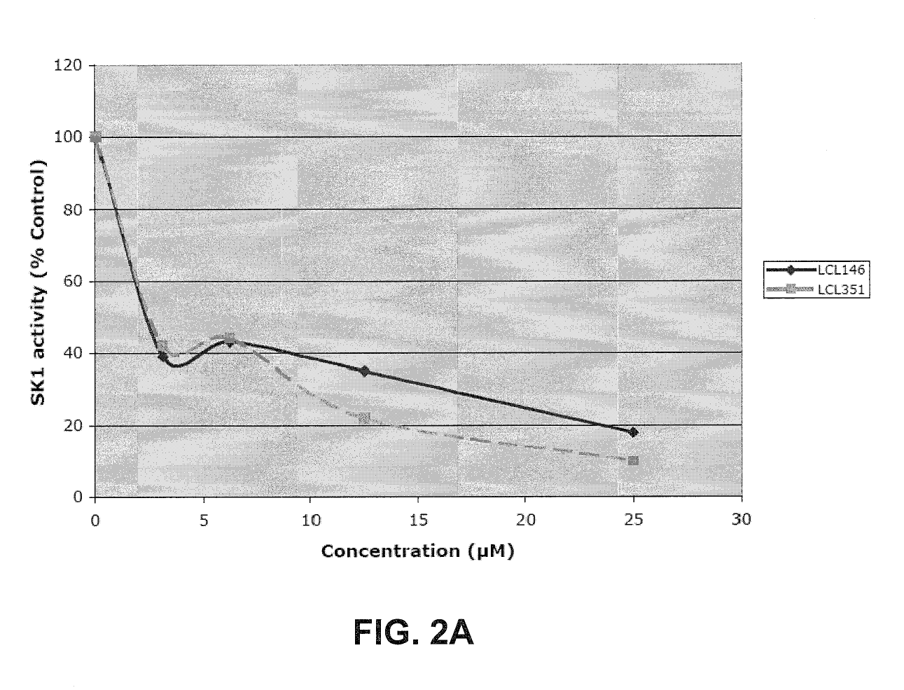

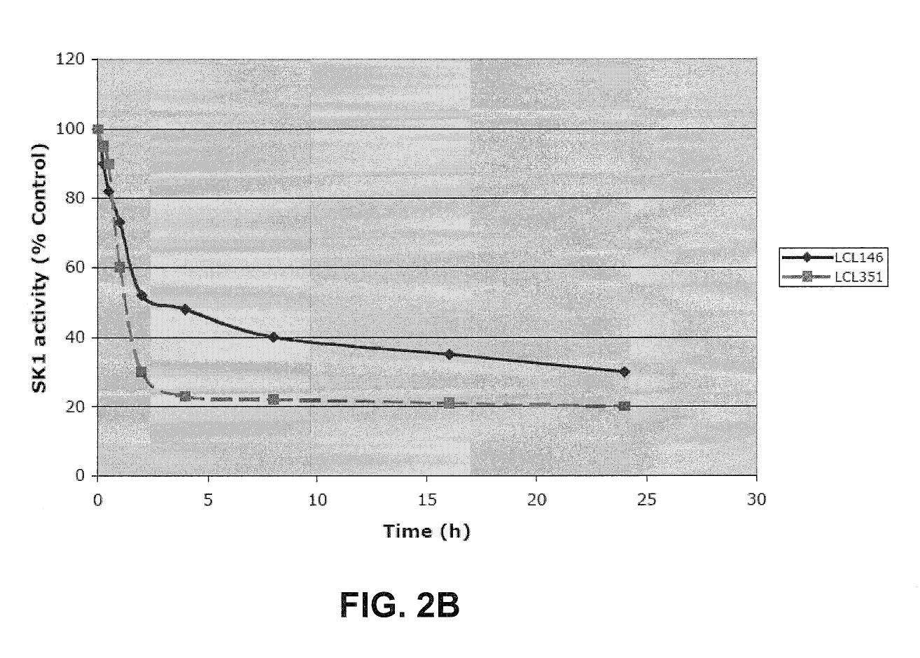

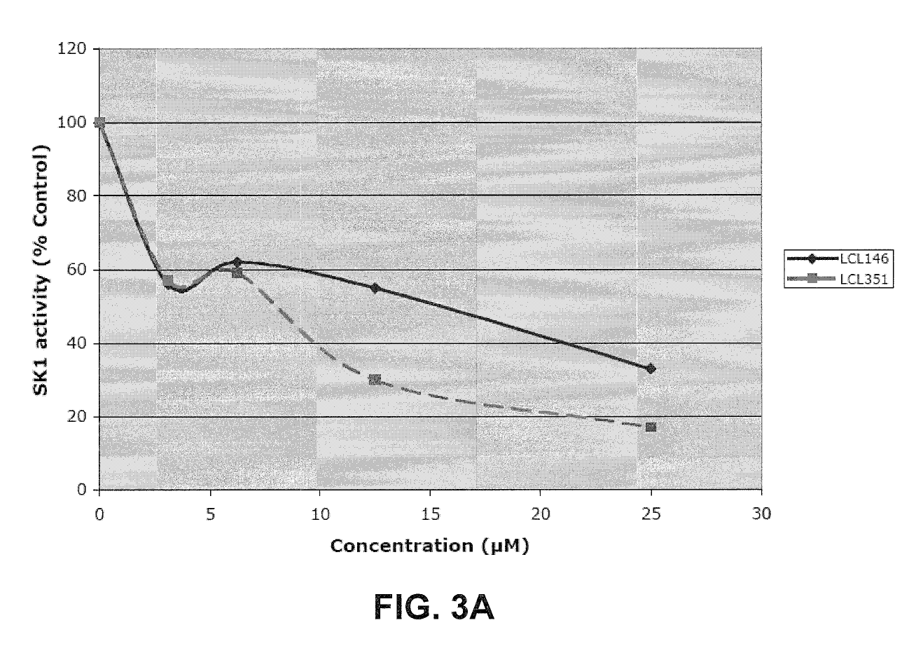

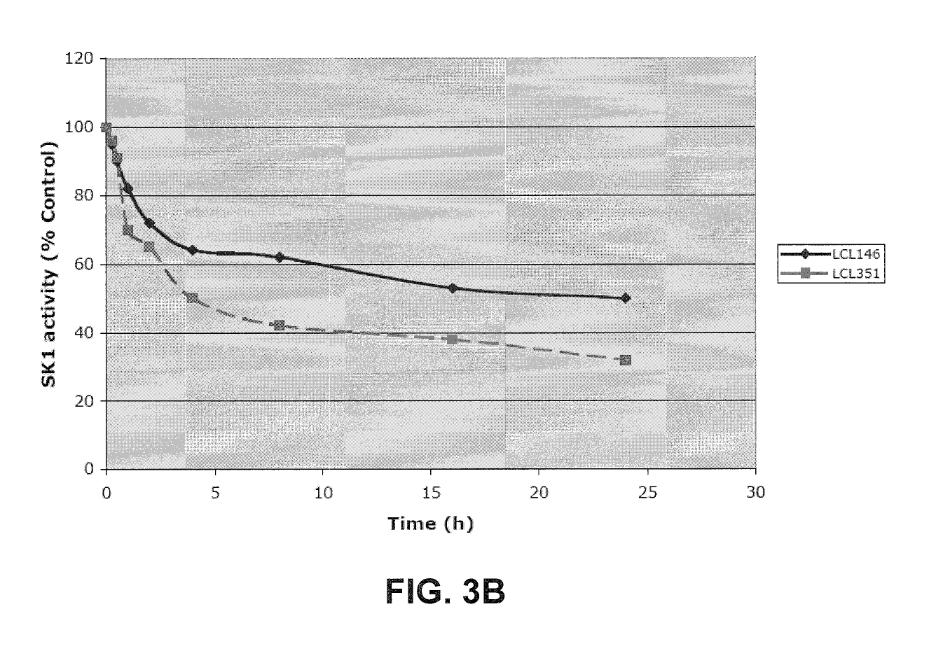

The inhibitory effects of LCL351 and LCL146 on SK1 activity in human non small cell lung cancer (NSCLC) cell lines is also shown in FIGS. 2A and 2B (concentration dependent pretreatment with LCL351 or LCL146 for 24 hours) and in FIGS. 3A and 3B (treatment with 12.5 μM LCL146 or LCL351 over time). Experiments were performed using H650 and H2405 lung cancer cells and treating them with the indicated guandino-sphingosine inhibitor. Cells were then harvested and cellular SK activity was determined.

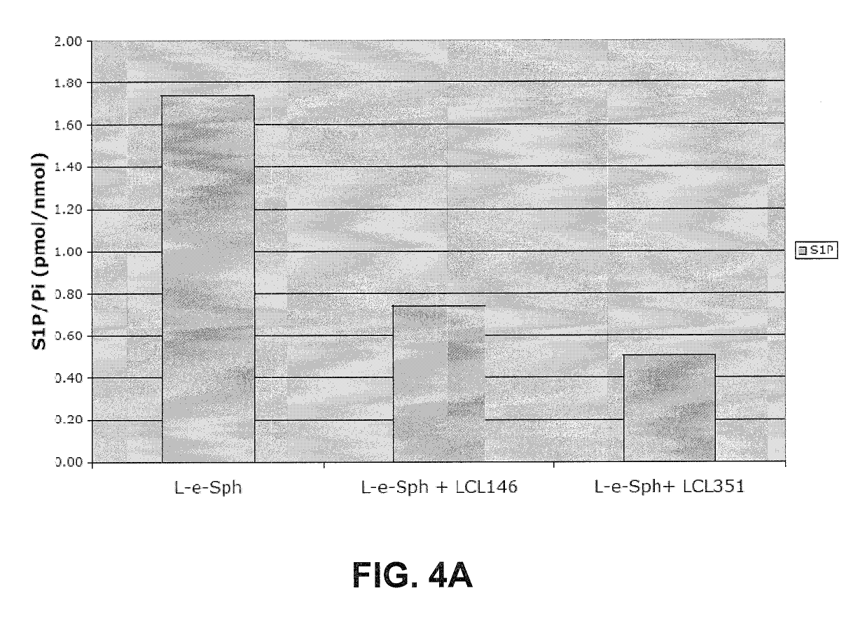

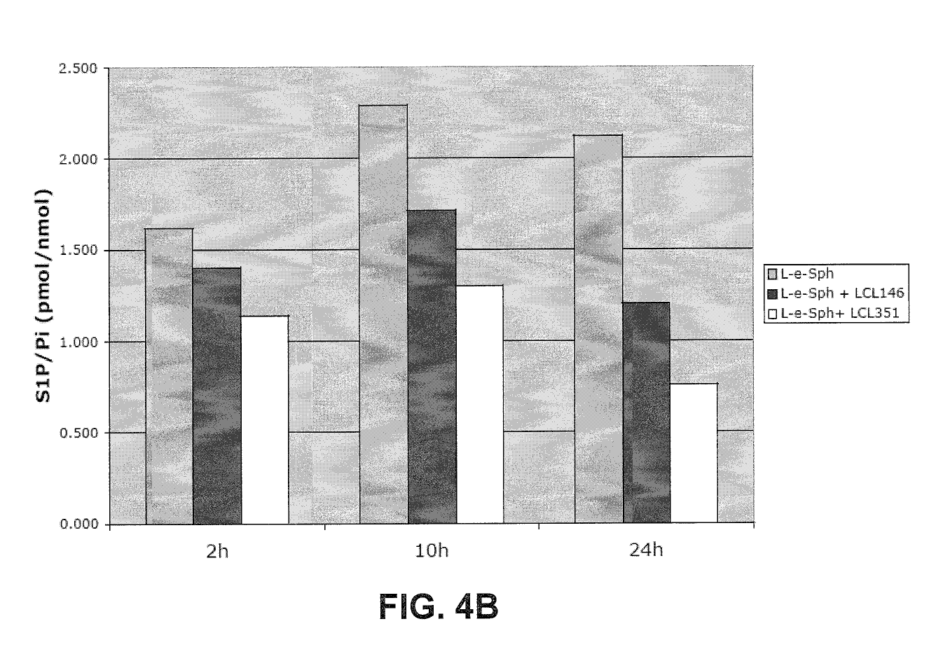

The inhibitory effects of LCL351 and LCL146 on S1P formation from exogenously added Sph (i.e., L-e-Sph) in MCF7 cells was also determined. MCF7 cells were pretreated for 24 hours with the inhibitory compounds (10 μM) and then for 2 hours with Sph. See FIG. 4A. Alternatively, the cells were treated simultaneously with inhibitor (10 μM) and Sph, and activity was determined at 2, 10, and 24 hours. See FIG. 4B.

### Example 3

### Effects of Guanidino-Sphingosines on Cancer Cells

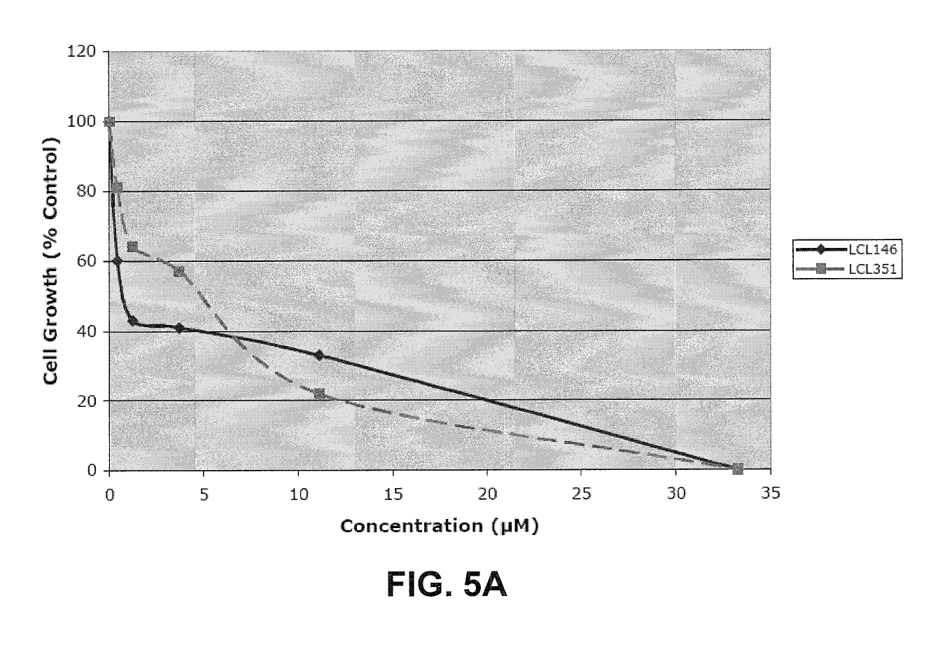

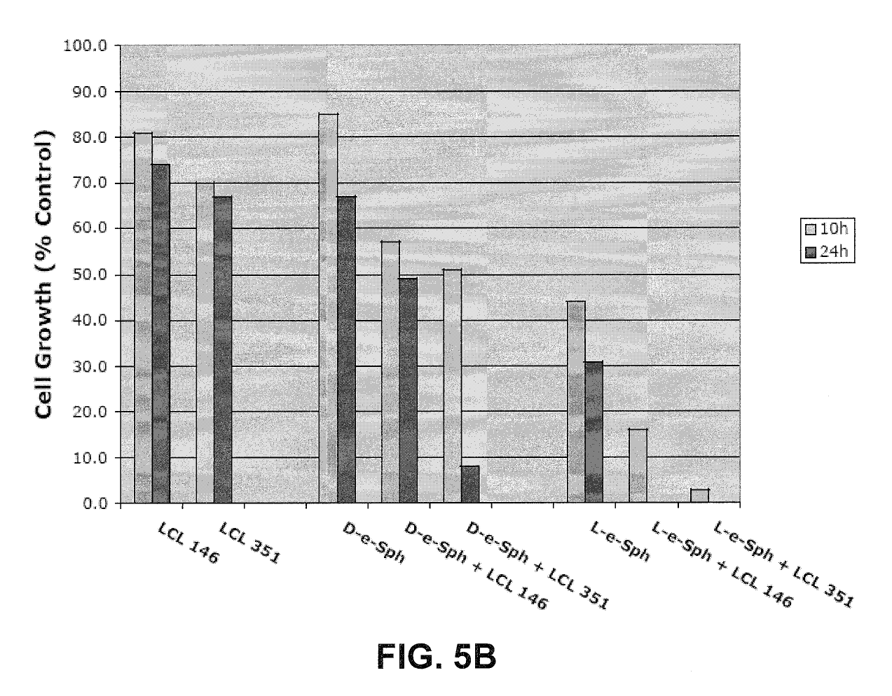

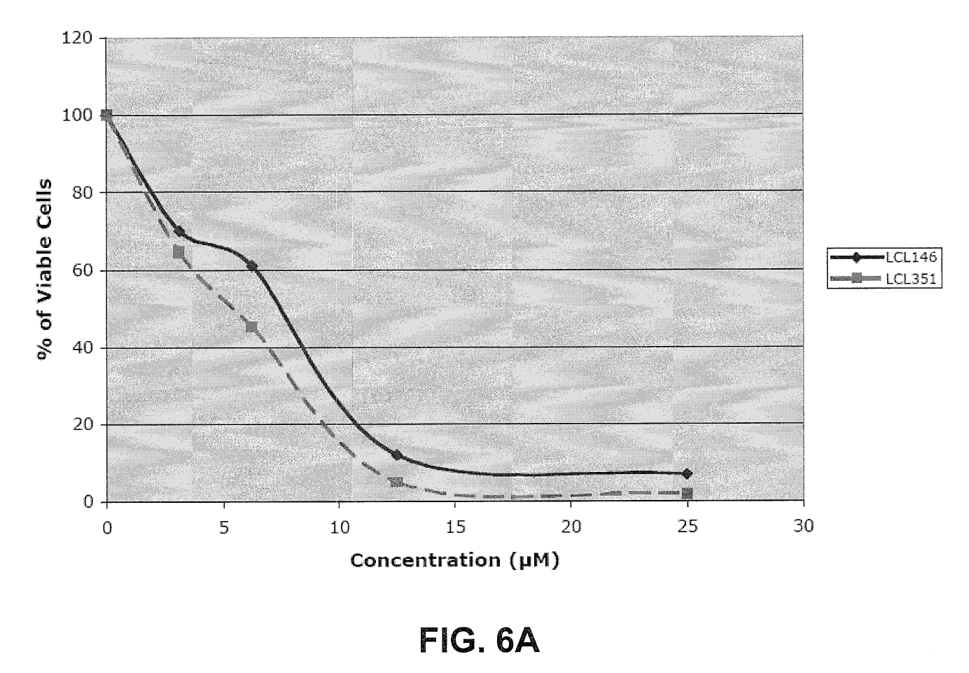

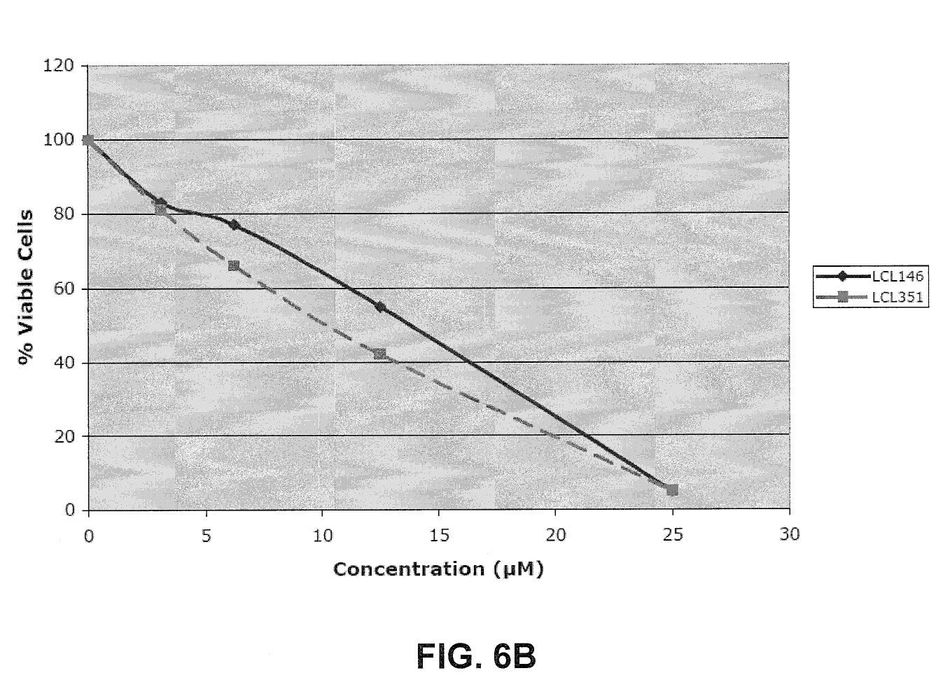

The guanidino-sphingosines were administered to mammalian cells capable of undergoing differentiation in amounts (low μM concentration) effective to induce differentiation. The inhibitory effects of LCL351 and LCL146 in MCF7 breast cancer cells are shown in FIG. 5A. MCF7 cells were seeded at a density of ˜50% (corresponding to 1×10⁶ cells) in 10 mL of 10% fetal calf serum (FCS) and, after an overnight incubation, were treated with the guanidino-sphingosine compound (0-50 μM in ethanol). Changes in cell numbers after 48 hours were determined and expressed as a percentage of the untreated controls. Enhanced inhibitory effects of LCL146, LCL351 (10 μM) and D-e- and L-e-Sph on MCF7 cell growth at 10 and 24 hours is shown in FIG. 5B. The effects of LCL146 and LCL351 on cell viability of human NSCLC cell lines after 48 hours is shown in FIGS. 6A (H650 cells) and 6B (H2405 cells).

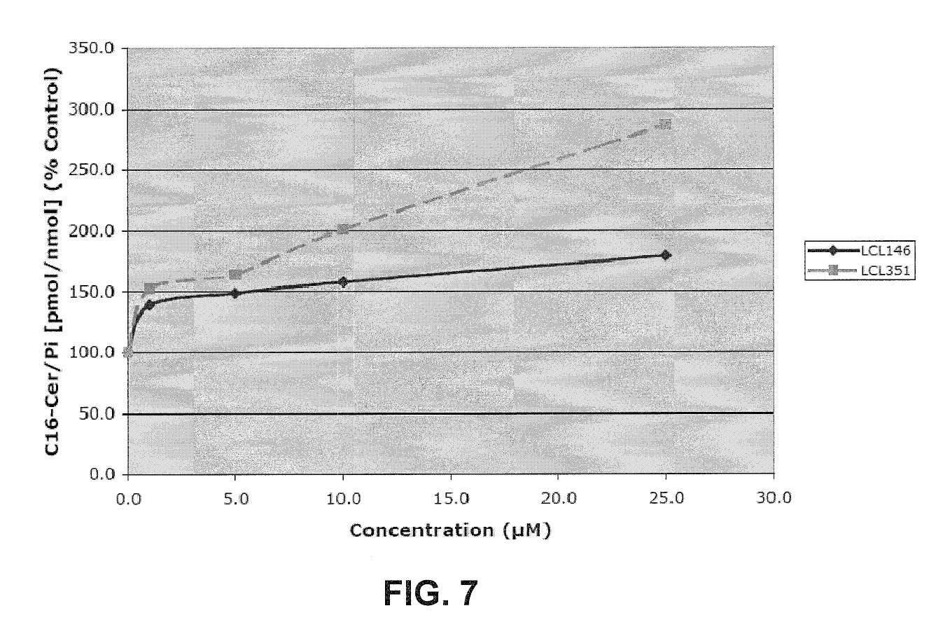

The administration of guanidino-sphingosines to mammalian cells at concentrations capable of inhibiting SK1 or SK2 were also found to increase the level of endogenous Cer in the cells. FIG. 7 shows the elevation in endogenous C₁₆-ceraminde levels in MCF7 cells following 24 hour treatment with LCL146 (darkly shaded diamonds) or LCL351 (lightly shaded squares) at concentrations between 0-25 μM.

### Example 4

### Effects of Guanidino-Sphingosines on Cancer Metastasis

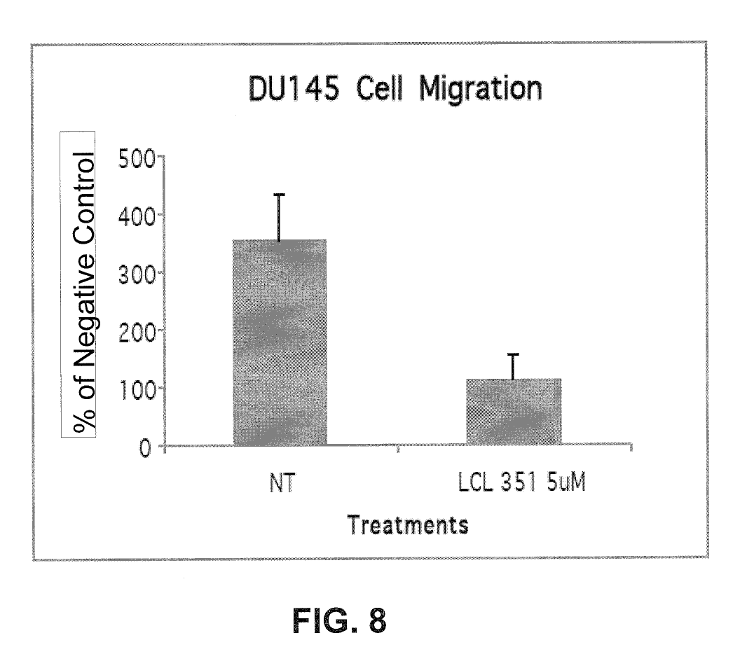

The presently disclosed guanidino-sphingosines were determined to be capable of inhibiting cell migration of cancer cells overexpressing acid ceramidase. The BD™ FALCON™ (Becton Dickinson and Company, Franklin Lakes, N.J., United States of America) migration assay (using FLUOROBLOK™ polyethylene terphthalate (PET) inserts (8.0 μm)) was performed with prostate cancer (DU145) cells over-expressing acid ceramidase. Cells were pretreated with 5 μM LCL351 overnight. After labeling the cells with carboxyfluorescein diacetate succinimidyl ester fluorscent dye (CFMDA), the cells were seeded on the top well of BDTM FLUOROBLOK™ PET membrane collagen-coated inserts. The negative control consisted of serum free media loaded in the bottom well and 10% FCS-enriched media was used as the positive control. After 16/24 hours incubation, the plate was read from the bottom (485/530 nm) and fluorescence was quantified. The effects of LCL351 are shown in FIG. 8, illustrating that the compound decreases the migration rate of DU145 cells.

It will be understood that various details of the presently disclosed subject matter may be changed without departing from the scope of the presently disclosed subject matter. Furthermore, the foregoing description is for the purpose of illustration only, and not for the purpose of limitation.

What is claimed is:

1. A compound of Formula (I): 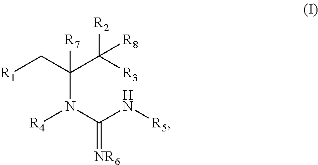
wherein:
R₁ is selected from the group consisting of H, alkyl, OH, (═O), (═N—OH), and SH;
R₂ is selected from the group consisting of H, alkyl, OH, SH, (═O), and (═N—OH);
R₃ is selected from the group consisting of H, alkyl, substituted alkyl, unsaturated alkyl, aryl, substituted aryl, aralkyl, OH, alkoxyl, aryloxyl, aralkoxyl, amino, aminoalkyl, an aminoacid moiety, and a peptidyl moiety;
R₄ and R₅ are independently selected from the group consisting of H, C(═NH)—NH₂, acyl, alkyl, substituted alkyl, aralkyl, aryl, and substituted aryl;
R₆ is selected from the group consisting of H, CN, alkyl, substituted alkyl, aryl, substituted aryl, aralkyl, and acyl;
R₇ is selected from the group consisting of H, alkyl, substituted alkyl, aralkyl, aryl, and substituted aryl; and
R₈ is present or absent, and when present is selected from the group consisting of H, alkyl, substituted alkyl, aralkyl, aryl, and substituted aryl;
or a pharmaceutically acceptable salt thereof.

2. The compound of claim 1, wherein R₁ is selected from OH and H.

3. The compound of claim 1, wherein R₂ is selected from OH, (═O), and H.

4. The compound of claim 1, wherein R₁ and R₂ are each OH.

5. The compound of claim 1, wherein R₃ is selected from C₁₅ fully saturated alkyl, C₁₅ hydroxy-substituted alkyl, C₁₅ alkenyl, C₁₅ alkynyl, aralkyl, phenyl, and H.

6. The compound of claim 1, wherein R₄ is H.

7. The compound of claim 1, wherein R₅ is H.

8. A pharmaceutically acceptable salt of a compound of claim 1, wherein the pharmaceutically acceptable salt of the compound of claim 1 has a structure of Formula (Ia): 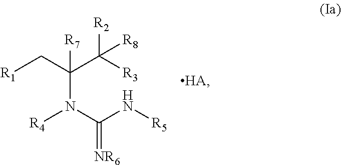
wherein:
R₁ is selected from the group consisting of H, alkyl, OH, (═O), (═N—OH), and SH;
R₂ is selected from the group consisting of H, alkyl, OH, SH, (═O), and (═N—OH);
R₃ is selected from the group consisting of H, alkyl, substituted alkyl, unsaturated alkyl, aryl, substituted aryl, aralkyl, OH, alkoxyl, aryloxyl, aralkoxyl, amino, aminoalkyl, an aminoacid moiety, and a peptidyl moiety;
R₄ and R₅ are independently selected from the group consisting of H, C(═NH)—NH₂, acyl, alkyl, substituted alkyl, aralkyl, aryl, and substituted aryl;
R₆ is selected from the group consisting of H, CN, alkyl, substituted alkyl, aryl, substituted aryl, aralkyl, and acyl;
R₇ is selected from the group consisting of H, alkyl, substituted alkyl, aralkyl, aryl, and substituted aryl;
R₃ is present or absent, and when present is selected from the group consisting of H, alkyl, substituted alkyl, aralkyl, aryl, and substituted aryl; and
A is selected from the group consisting of bromide, chloride, sulfate, acetate, dichloroacetate, benzoate, and tartrate.

9. The pharmaceutically acceptable salt of claim 8, wherein A is chloride.

10. The compound of claim 1, wherein the compound is selected from the group consisting of D-erythro-2-guanidino-sphingosine hydrochloride and L-erythro-2-guanidino-sphinogosine hydrochloride.

11. A method of inhibiting sphingosine kinase, the method comprising contacting a sample comprising sphingosine kinase with an effective amount of a compound of Formula (I): 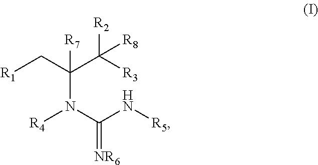
wherein:
R₁ is selected from the group consisting of H, alkyl, OH, (═O), (═N—OH), and SH;
R₂ is selected from the group consisting of H, alkyl, OH, SH, (═O), and (═N—OH);
R₃ is selected from the group consisting of H, alkyl, substituted alkyl, unsaturated alkyl, aryl, substituted aryl, aralkyl, OH, alkoxyl, aryloxyl, aralkoxyl, amino, aminoalkyl, an aminoacid moiety, and a peptidyl moiety;
R₄ and R₅ are independently selected from the group consisting of H, C(═NH)—NH₂, acyl, alkyl, substituted alkyl, aralkyl, aryl, and substituted aryl;
R₆ is selected from the group consisting of H, CN, alkyl, substituted alkyl, aryl, substituted aryl, aralkyl, and acyl;
R₇ is selected from the group consisting of H, alkyl, substituted alkyl, aralkyl, aryl, and substituted aryl; and
R₅ is present or absent, and when present is selected from the group consisting of H, alkyl, substituted alkyl, aralkyl, aryl and substituted aryl;
or a pharmaceutically acceptable salt thereof.

12. The method of claim 11, wherein the compound selectively inhibits sphingosine kinase 1.

13. The method of claim 11, wherein the compound is essentially completely water soluble.

14. The method of claim 11, wherein the sample is an in vitro sample.

15. The method of claim 11, wherein the compound inhibits sphingosine kinase with a 50% inhibitory concentration (IC₅₀) of less than about 1.0 μM.

16. The method of claim 15, wherein the compound inhibits sphingosine kinase with an IC₅₀ of less than about 0.4 μM.

17. The method of claim 11, wherein R₁ is selected from OH and H.

18. The method of claim 11, wherein R₂ is selected from OH, (═O), and H.

19. The method of claim 11, wherein R₁ and R₂ are each OH.

20. The method of claim 11, wherein R₃ is selected from C₁₅ fully saturated alkyl, C₁₅ hydroxy-substituted alkyl, C₁₅ alkenyl, C₁₅ alkynyl, aralkyl, phenyl, and H.

21. The method of claim 11, wherein R₄ is H.

22. The method of claim 11, wherein R₅ is H.

23. The method of claim 11, wherein the compound is contacted to the sample in the form of a pharmaceutically acceptable salt.

24. The method of claim 23, wherein the pharmaceutically acceptable salt is a hydrochloride salt.

25. The method of claim 11, wherein the compound is selected from the group consisting of D-erythro-2-guanidino-sphingosine hydrochloride and L-erythro-2-guanidino-sphinogosine hydrochloride.

26. A method of treating or preventing a disease or disorder associated with undesirable ceramidase or sphingosine kinase activity in a subject, the method comprising administering to the subject an effective amount of a compound of Formula (I): 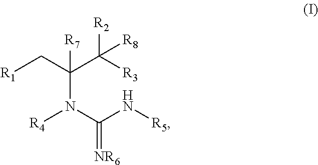
wherein:
R₁ is selected from the group consisting of H, alkyl, OH, (═O), (═N—OH), and SH;
R₂ is selected from the group consisting of H, alkyl, OH, SH, (═O), and (═N—OH);
R₃ is selected from the group consisting of H, alkyl, substituted alkyl, unsaturated alkyl, aryl, substituted aryl, aralkyl, OH, alkoxyl, aryloxyl, aralkoxyl, amino, aminoalkyl, an aminoacid moiety, and a peptidyl moiety;
R₄ and R₅ are independently selected from the group consisting of H, C(═NH)—NH₂, acyl, alkyl, substituted alkyl, aralkyl, aryl, and substituted aryl;
R₆ is selected from the group consisting of H, CN, alkyl, substituted alkyl, aryl, substituted aryl, aralkyl, and acyl;
R₇ is selected from the group consisting of H, alkyl, substituted alkyl, aralkyl, aryl, and substituted aryl; and
R₈ is present or absent, and when present is selected from the group consisting of H, alkyl, substituted alkyl, aralkyl, aryl and substituted aryl;
or a pharmaceutically acceptable salt thereof, wherein said compound inhibits sphingosine kinase.

27. The method of claim 26, wherein the compound selective inhibits sphingosine kinase 1.

28. The method of claim 26, wherein the compound is essentially completely water soluble.

29. The method of claim 26, wherein the disease or disorder is selected from the group consisting of cancer, cancer metastasis, atherosclerosis, stenosis, inflammation, an immunological disorder, asthma, atopic dermatitis, wound healing and other proliferative diseases.

30. The method of claim 26, wherein the subject is a mammalian subject.

31. The method of claim 26, wherein the compound of Formula (I) is administered to the subject in a pharmaceutical formulation comprising the compound of Formula (I) and a pharmaceutically acceptable carrier.

32. A method of treating cancer, the method comprising administering to a subject in need of treatment thereof an effective amount of a compound of Formula (I): 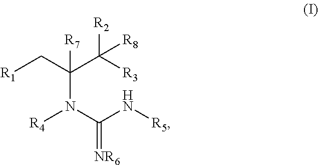
wherein:
R₁ is selected from the group consisting of H, alkyl, OH, (═O), (═N—OH), and SH,
R₂ is selected from the group consisting of H, alkyl, OH, SH, (═O), and (═N—OH);
R₃ is selected from the group consisting of H, alkyl, substituted alkyl, unsaturated alkyl, aryl, substituted aryl, aralkyl, OH, alkoxyl, aryloxyl, aralkoxyl, amino, aminoalkyl, an aminoacid moiety, and a peptidyl moiety;
R₄ and R₅ are independently selected from the group consisting of H, C(═NH)—NH₂, acyl, alkyl, substituted alkyl, aralkyl, aryl, and substituted aryl;
R₆ is selected from the group consisting of H, CN, alkyl, substituted alkyl, aryl, substituted aryl, aralkyl, and acyl;
R₇ is selected from the group consisting of H, alkyl, substituted alkyl, aralkyl, aryl, and substituted aryl; and
R₈ is present or absent, and when present is selected from the group consisting of H, alkyl, substituted alkyl, aralkyl, aryl, and substituted aryl;
or a pharmaceutically acceptable salt thereof.

33. The method of claim 32, wherein the cancer is selected from the group consisting of breast cancer, prostate cancer, and non small cell lung cancer.

34. The method of claim 32, wherein the subject is a mammalian subject.

35. The method of claim 32, wherein the compound of Formula (I) is administered in a pharmaceutical formulation comprising the compound of Formula (I) and a pharmaceutically acceptable carrier.

36. The method of claim 32, wherein R₁ is selected from OH and H.

37. The method of claim 32, wherein R₂ is selected from OH, (═O), and H.

38. The method of claim 32, wherein R₁ and R₂ are each OH.

39. The method of claim 32, wherein R₃ is selected from C₁₅ fully saturated alkyl, C₁₅ hydroxy-substituted alkyl, C₁₅ alkenyl, C₁₅ alkynyl, aralkyl, phenyl, and H.

40. The method of claim 32, wherein R₄ is H.

41. The method of claim 32, wherein R₅ is H.

42. The method of claim 32, wherein the compound is administered in the form of a pharmaceutically acceptable salt.

43. The method of claim 42, wherein the pharmaceutically acceptable salt is a hydrochloride salt.

44. The method of claim 32, wherein the compound is selected from the group consisting of D-erythro-2-guanidino-sphingosine hydrochloride and L-erythro-2-guanidino-sphinogosine hydrochloride.

45. The method of claim 32, wherein treating cancer comprises preventing the metastasis of a cancer.
